(19) United States
(12) Patent Application Publication
(10) Pub. No.: US 2024/0115608 A1
(43) Pub. Date: Apr. 11, 2024

(54) IMMUNE RECEPTORS WITH SYNTHETIC CO-STIMULATORY DOMAINS

(71) Applicant: The Regents of the University of California, Oakland, CA (US)

(72) Inventors: Wendell A. LIM, San Francisco, CA (US); Kyle DANIELS, San Francisco, CA (US)

(21) Appl. No.: 18/274,979

(22) PCT Filed: Feb. 7, 2022
(86) PCT No.: PCT/US2022/015513

§ 371 Date: Jul. 28, 2023

Related U.S. Application Data
(60) Provisional application No. 63/279,578, filed on Nov. 15, 2021.
(60) Provisional application No. 63/148,056, filed on Feb. 10, 2021.

(51) Int. Cl. A61K 35/17; A61K 39/00; A61P 35/00; C07K 14/725; C07K 16/28

(52) U.S. Cl. CPC A61K 35/17; A61K 39/4611; A61K 39/4631; A61K 39/464411; A61P 35/00; C07K 14/7051; C07K 16/2809; A61K 2239/15; A61K 2239/21

(57) ABSTRACT

An engineered immune receptor (e.g., a chimeric antigen receptor (CAR) or chimeric costimulatory receptor (CCR)) that contains one or more short linear motifs that bind to other intracellular signaling proteins are provided, as well as nucleic acids encoding the same, cells that contain the same and methods of use. Examples of such motifs include a PLCγ1-binding motifs and TRAF binding motifs, but other motifs may be used. These motifs are thought to recruit other proteins to the engineered immune receptor, thereby altering cellular responses.

### CROSS-REFERENCING

This application claims the benefit of U.S. provisional application Ser. No. 63/279,578, filed on Nov. 15, 2021, and 63/148,056, filed on Feb. 10, 2021, which applications are incorporated by reference herein.

### GOVERNMENT SUPPORT

This invention was made with government support under grant no. RO1 CA220257 awarded by The National Institutes of Health. The government has certain rights in the invention.

### INTRODUCTION

Chimeric antigen receptors (CARs) have demonstrated the power of synthetic signaling receptors as tools to reprogram immune cells to carry out novel therapeutic functions, such as selective killing of tumor cells (1). The antitumor efficacy of CARs is strongly modulated by the signaling domains that they contain. Current clinically approved CARs contain a core TCR signaling domain from CD3ζ ((containing ITAM motifs that recruit the kinase ZAP70) (2-4), along with a costimulatory signaling domain from either the CD28 (5, 6) or 4-1BB (7) costimulatory immune receptors (8-10). The costimulatory domains are themselves composed of multiple signaling motifs, short peptides that bind to specific downstream signaling proteins, often through modular protein interaction domains (e.g. SH2, SH3, or other domains (11, 12)). Such peptide signaling motifs (referred to as linear motifs) are the fundamental building blocks of most signaling receptors. The constellation of signaling proteins recruited by a particular array of signaling motifs is postulated to shape the distinct cellular response. For example, in CARs, the 4-1BB costimulatory domain which contains TRAF binding motifs, leads to increased T cell memory and persistence; the CD28 costimulatory domain, which contains PI3K, Grb2, and Lck binding motifs, is associated with more effective T cell killing, but reduced long-term persistence (13). Thus, signaling motifs can be thought of as the “words” that are used to compose the phenotypic “sentences” of signaling domains.

A major goal in synthetic biology is to predictably generate new cell phenotypes by altering receptor composition. For example, in cancer immunotherapy, a general goal is to enhance T cell anti-tumor cytotoxicity as well as maintenance of a stem-like state associated with longer-term T cell persistence. Such a phenotype is associated with effective and durable tumor clearance (higher stemness is correlated with more resistance to T cell exhaustion). In recent work, libraries of costimulatory domains have been screened for improved phenotypes (14-16). Such studies, however, have focused on screening intact costimulatory domains from natural immune receptors (i.e., alternative pre-existing “sentences”).

New signaling domains with better properties are therefore needed.

### SUMMARY

An engineered immune receptor (e.g., a chimeric antigen receptor (CAR) or chimeric costimulatory receptor (CCR)) that contain short linear motifs that bind to other intracellular signaling proteins and combinations thereof are provided, as well as nucleic acids encoding the same, cells that contain the same and methods of use. Examples of the signaling proteins and exemplary motifs to which they bind are shown in table 1. These motifs are thought to recruit other proteins to the immune receptor, thereby altering cellular responses.

In some embodiments, the linear motif may be a PLCγ1-binding motif although, in other embodiments, another motif may be used, e.g., a motif that binds to Lck, TRAF2, TRAF1, TRAF6, Pellino protein, TIFA, PI3K, Grb2, GADS, Vav1, SHP-1 or SHP2 etc. See Table 1. In some cases, the PLCγ1-binding motif may be of the sequence (Y)[AFILVWY]×[AFILVWY], where (Y) is a tyrosine followed by any choice of AFILVWY, then any amino acid, then any choice of AFILVWY although it is understood that not all PLCγ1 binding sequences fit this consensus sequence, and some sequences that fall into this consensus sequence bind the SH2 domains of other proteins and not of PLCγ1. In these embodiments, it is thought that the first Y of the motif may be phosphorylated in the cell, thereby recruiting PLCγ1 to the immune receptor. Examples of PLCγ1-binding motifs that could be used include YLVV (SEQ ID NO: 2) motif from LAT, YIIP (SEQ ID NO: 3) from platelet-derived growth factor receptor beta, YLIP (SEQ ID NO: 4) & YLRV (SEQ ID NO: 5) of EGFR, and YLVP (SEQ ID NO: 6) of ERBB2. The binding sites can be combined in any way. For example, an immune receptor may comprise a PLCγ1-binding motif as well as a TRAF (TRAF2, TRAF1 or TRAF6)-binding motif.

In some embodiments, the addition of the motif to a CAR makes T cells that are activated through the more stemlike (i.e., with reduced T cell exhaustion). As such, immune cells that are activated via engineered immune receptors that have such a linear motif may be clinically better than immune cells that are activated via engineered immune receptors that that do not have such a linear motif.

In addition to CARs, these motifs can also be used in place of or in addition to a conventional costimulatory domain in a chimeric costimulatory receptor (CCR), which are generally used in conjunction with a CARs or TCRs to enhance or modulate therapeutic

### BRIEF DESCRIPTION OF THE DRAWINGS

The skilled artisan will understand that the drawings, described below, are for illustration purposes only. The drawings are not intended to limit the scope of the present teachings in any way.

FIGS. 1A-1E. CAR costimulatory domains with novel signaling motif combinations generate diverse cell fates with decoupled cytotoxicity and stemness. FIG. 1A, A diverse set of proteins involved in T cell signaling are recruited by signaling motifs in the library parts. FIG. 1B, Description of library parts used in combinatorial library. Each part is 16-18 amino acids including the signaling motif(s) (SEQ ID NOs: 6-19; from top to bottom and left to right) and flanking sequence. Phospho-tyrosines are shown in bold. FIG. 1C, New combinations of signaling motifs create novel CAR signaling programs that control T cell phenotype. FIG. 1D, Schematics of a CD19 CAR with variable signaling domains. FIG. 1E, CAR T cells with novel signaling motif combinations produce a broad range of cytotoxicity and stemness. Several combinations produce cytotoxicity and stemness comparable to or exceeding that of CD28 and 4-1BB. Errors for Nalm 6 survival, and stem-like IL7Rα+/KLRG1− population in E were estimated by calculating the average s.e.m. for 7 CAR constructs with internal duplicates in the array.

FIGS. 2A-2D. Neural networks decode the combinatorial language of signaling motifs to predict cytotoxicity and sternness of novel motif combinations. FIG. 2A, Array data were subdivided in datasets to train and test neural networks that were subsequently used to predict the cytotoxicity and stemness of 2379 CARs. FIG. 2B, Schematic of neural network used to predict CAR T cell phenotype. FIG. 2C, Neural networks trained on array data predict the cytotoxicity and sternness of CARs in the training sets (black) and the withheld test sets (pink). FIG. 2D, Trained neural networks were used to predict the cytotoxicity and sternness of 2379 CARs containing 1-3 variable signaling motifs. Predictions represent the mean for n=10 neural networks with different hyperparameters.

FIGS. 3A-3D. Distribution analysis quantifies elements of linear motif language to extract design parameters for signaling domains. FIG. 3A, The distribution of library parts throughout CARs in the ranked library reflects effects of signaling motifs on phenotype. Activating motifs are found in CARs with higher rank and inhibitory motifs are found in CARs with lower rank. The three lines within the distributions represent mean predictions±s.e.m. calculated from n=10 neural networks. FIG. 3B, CARs containing pairs of motifs that recruit TRAFs (P9 and P10) or PLCγ1 (P1) promote robust cytotoxicity and stemness. FIG. 3C, The effects of signaling motifs on phenotype are position-dependent. FIG. 3D, P1 is predicted to promote cytotoxicity best at position k, while P6 is predicted to inhibit cytotoxicity best at position k.

FIGS. 4A-4E. Neural networks accurately predict that PLCγ1 binding motifs improve the cytotoxicity and stemness of 4-1BB-ζ but not CD28-ζ. FIG. 4A, Library parts that share consensus signaling motifs with 4-1BB and CD28 costimulatory domains were used to predict the effect of adding at P1 to 4-1BB and CD28. FIG. 4B, Addition of 1 or 2 copies of P1 improved in vitro cytotoxicity and sternness of 4-1BB-ζ but not CD28-ζ. Cytotoxicity and stemness were assessed after 4 challenges with Nalm 6 cells. Data are mean±s.e.m. of n=3-5 replicates. FIG. 4C, NSG mice were injected intravenously with 0.5×10⁶ Nalm 6 cells, and then injected intravenously with 3×10⁶ CAR+ T cells on day 4. CAR T cells with 4-1BB-P1-P1-ζ showed improved early tumor control relative to 4-1BB-ζ. Traces in C are median luminescence±95% confidence interval. FIG. 4D, Costimulatory PLCγ1 signaling is redundant to signaling provided by PI3K and Grb2, but complementary to TRAF signaling. FIG. 4E, Addition of P1 to 4-1BB-ζ induced modest changes in Akt phosphorylation—which is not downstream of PLCγ1 signaling—relative to the changes in ERK1/2 and NFkB phosphorylation—which are downstream of PLCγ1 signaling. Data for FIG. 4D are mean and standard deviation of n=3 replicates.

FIGS. 5A-5L. CARs with novel signaling motif combinations generate diverse T cell outputs of proliferation, memory formation, and degranulation in a pooled screen. FIG. 5A, Workflow for pooled screening of pooled combinatorial CAR library. FIG. 5B, Timeline for pooled combinatorial CAR library screen. FIG. 5C, 2378 of the 2379 CAR constructs were detected by sequencing the pooled plasmid library, and over 1700 CAR constructs were detected by sequencing DNA from GFP+CD4 and CD8 CAR T cells. FIG. 5D, CAR T cell proliferation calculated by Log 2 fold change in CAR construct frequency 9 days after initial stimulation relative to the starting populations indicates the constructs in the library promote differing degrees of T cell proliferation. FIGS. 5E-5H, Select populations of central memory cells and degranulating cells were isolated by FACS according to the gates shown. Isolated cells were later sequenced. FIGS. 5I-5L, CAR T cell memory formation and degranulation were calculated by Log 2 fold change in CAR construct frequency in FACS-isolated select populations on day 9 relative to total populations on day 9. Constructs in the library promote differing degrees of central memory formation and degranulation. All enrichment plot data are mean±s.e.m. of n=3 replicates.

FIGS. 6A-6D. CAR costimulatory domains with novel signaling motif combinations generate diverse cell fates. FIG. 6A, CAR T cells with novel signaling motif combinations generate a broad range naïve+central memory populations (quantified by CD62L expression). FIG. 6B, CD62L expression is positively correlated with cytotoxicity (r²=0.65). Errors for Nalm 6 survival, and % CD62L+ population in B were estimated by calculating the average s.e.m. for 7 CAR constructs in the array with internal duplicates. FIG. 6C, CARs containing P1 and P10 generate high cytotoxicity and stemness when combined such that P1 is distal from the membrane, but generate reduced cytotoxicity and sternness when not combined or when P1 is not distal from the membrane. FIG. 6D. P1-P10-P2-ζ generates low cytotoxicity and high sternness.

FIGS. 7A-7C. Several common machine learning algorithms fail to predict CAR T cell phenotype. FIG. 7A, Array data were subdivided in datasets to train and test various machine learning algorithms. FIGS. 7B-7C, Linear regression, nearest neighbors, random forest, gradient boosted trees, and decision tree algorithms were used to predict cytotoxicity (FIG. 7B) and stemness (FIG. 7C) resulting from various combinations of library parts.

FIGS. 8A-8B. Distribution analysis quantifies elements of linear motif language to extract design parameters for signaling domains. FIG. 8A, Heatmaps of mean ranked percentile quantify the overall effects of library parts on CAR T cell cytotoxicity and sternness. FIG. 8B, Heatmaps of mean ranked percentile quantify the position-dependent effects of library parts on CAR T cell cytotoxicity and sternness.

FIGS. 9A-9J. Distribution analysis quantifies contributions of library parts to CAR T cell proliferation in a pooled screen. FIG. 9A, Example percentile distributions for CARs that contain parts with various effects on CAR T cell phenotype. FIG. 9B, Percentile distributions from pooled screening demonstrate the varied effects of library parts on CD4+ T cell proliferation. The three lines within the distributions represent mean±s.e.m. for n=3 pooled library screens. FIG. 9C, Heatmap of mean ranked percentile quantifies the overall effects of library parts on CAR T cell proliferation measured in pooled screens. FIG. 9D, Example percentile distributions for CARs that contain individual parts or pairs parts. FIG. 9E, The most activating and most inhibitory pairs calculated using the means of percentile distributions. FIG. 9F, Mean ranked percentile for all pairs in the library. FIG. 9G, CAR schematic depicting positions i, j, and k in variable costimulatory domain. FIG. 9H, Percentile distributions from pooled screening demonstrate the position-dependent effects of P1 and P12 on CD4+ T cell proliferation. FIG. 9I, Heatmaps of mean ranked percentile quantify the position-dependent effects of library parts on CAR T cell proliferation measured in pooled screens. FIG. 9J, Heatmap of mean ranked percentile quantifies the overall effects of library parts on CAR T cell proliferation, central memory formation, and degranulation (a proxy for cytotoxicity) measured in pooled screens.

FIGS. 10A-10F. Neural networks accurately predict that PLCγ1 binding motifs improve the cytotoxicity and stemness of 4-1BB-ζ but not CD28-z. FIG. 10A, Schematics of signaling motifs in 4-1BB, CD28, and functionally similar library parts. FIGS. 10B-10C, In vitro assessment of the effect of adding one or two copies of P1 to 4-1BB and CD28 costimulatory domains. Cytotoxicity (FIG. 10B) and IL7Rα and KLRG1 expression (FIG. 10C) were assessed on day 9 after 4 challenges with Nalm 6 target cells. FIG. 10D, Tumor progression was monitored using bioluminescent imaging of Nalm 6 expressing the firefly luciferase (fLuc) transgene. Scales are normalized for all time points. FIGS. 10E-10F, Kinetics of phosphorylation upon stimulation of CAR T cells with Nalm 6 target cells, measured by flow cytometry. Kinetic traces represent mean and standard deviation for n=3 replicates.

FIGS. 11A-11C. (FIG. 11A) Addition of PLCγ1-binding motifs (P1) to the 4-1BB costimulatory domain increases in vitro killing and stemness of CAR T cells. 4-1BB variants with 1 or 2 copies of P1 between the 4-1BB and CD3ζ domains were made and tested for killing and stemness. Killing was assessed by calculating Nalm 6 target cell survival relative survival in wells containing untransduced (not engineered T cells). Stemness was assessed by gating on IL7Ra+/KLRG1− cells. Error bars represent mean and s.e.m. for n=3 or more measurements. (FIG. 11B) and (FIG. 11C): CAR T cells with 4-1BB-P1-P1-ζ signaling domains showed improved tumor control relative to 4-1BB-ζ. CAR T cells containing the aCD19 CAR with 4-1BB-ζ or 4-1BB-P1-P1-ζ signaling domains were injected into NSG mice to treat Nalm 6 leukemia. Nalm 6 tumor cells expressed a firefly luciferase (fLuc) transgene for tumor imaging. Traces in E are median Nalm 6 luminescence±95% confidence interval.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CHI) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Provided herein is an engineered immune receptor, comprising: an extracellular binding domain that binds to a protein on the surface of another cell; a transmembrane domain; and one or more (e.g., one, two, three, four, five or at least six) synthetic co-stimulatory motifs that bind to a protein listed in Table 1, or any combination thereof, in the intracellular part of the receptor. The core binding motifs for the proteins listed in table are generally quite short (e.g., 4-6 amino acids in length) and, as such, the synthetic co-stimulatory motif may be 4-50 amino acids in length (e.g., 10-20 amino acids) and contain a core binding motif. The receptor may have a combination of motifs that are not found in nature, for example. These sequences are generally much shorter than the domains that are typically included in receptors, which typically include intact natural domains such as 4-1BB, CD28, or the IL2Rb chain. In other words, the motifs used herein just contain the core sequence plus a few amino acids (e.g., up to 6, 7, 8, 9 or 10 amino acids either side of the motif).

In some embodiments, the receptor may contain one or more sequences shown in Table 1 (SEQ ID NO: 20-31 and 19 from top to bottom) or listed in the examples section below.

TABLE 1

| | | | Putative |
|---|---|---|---|
| Library | Sequence | | Binding |
| Part | (Core | Source | Partners |
| Name | Motif) | Protein | (domain) |
| [PLCγ1] | DYHNP | LAT | PLCγ1 (SH2) |
| | GYLVV | | |
| | LPDST | | |
| | P | | |
| [Lck] | LPTWS | CD4 | Lck (SH3) |
| | TPVQP | | |
| | MALIV | | |
| | LG | | |
| TRAF21] | GSNTA | CD40 | TRAF2 (TRAF.C), |
| | APYQE | | TRAF1 (TRAF.C) |
| | TLHGC | | |
| | Q | | |
| [TRAF22] | DDSLP | LMP1 | TRAF2 (TRAF-C), |
| | HPQQA | | TRAF1 (TRAF-C) |
| | TDDSG | | |
| | HES | | |
| [TRAF6] | KAPHA | CD40 | TRAF6 (TRAF.C) |
| | KQEPQ | | |
| | EINFP | | |
| | DDLP | | |
| [FHA] | GSGPG | IRAK1 | Pellino |
| | SRPTA | | protein |
| | VEGLA | | (FHA), |
| | LGSS | | TIFA (FHA) |
| [PI3K] | EELDE | Gab1 | PI3K (8M2) |
| | NYVPM | | |
| | NPNSP | | |
| | P | | |
| [Grb2] | EEGAP | LAT | Grb2 (SH2) |
| | DYENL | | |
| | QELNH | | |
| | P | | |
| [PI3K/Grb2] | LGSNQ | IL7R α | PI3K (SH2), |
| | EEEAY | Chain | Grt2 (SH2) |
| | VTMSS | | |
| | FYQNQ | | |
| [GADS] | PAPSI | SLP76 | GADS (6H3) |
| | DRSTK | | |
| | PPLDR | | |
| | SL | | |
| [VRV1] | LPMDT | SYK | VRV1 (SH2) |
| | EVYES | | |
| | PFADP | | |
| | EEIR | | |
| [SHP-1/2] | KPMAE | LAIR1 | SHP-1 (SH2), |
| | SITYA | | SHP-2 (SH2) |
| | AVARH | | |
| | SAG | | |
| [Linker] | SAGSA | Synthetic | Non-functional |
| | GSAGS | | control, |
| | AGSAG | | spacer |
| | SAG | | |

The addition of one more synthetic motif may improve cell performance, e.g., may make the cell more stem-like, may delay exhaustion, may make the cells more cytotoxic; may increase the production of pro-inflammatory cytokines (e.g., IL-2) may decrease expression of anti-inflammatory cytokines, may favor CD4+ T cell expansion, may alter Th1 and Th17 polarization, may stimulate CD8+ central memory T cell generation, may stimulate T cell persistence, may suppresses Treg development, may modulate Bcl-X(L) protein expression, may increase proliferation and secretion of pro-inflammatory Th1 cytokines, may increase or decrease IL-2, IFN-γ and GM-CSF production, etc. Specifically, an added motif recruit one or more other proteins to the engineered immune receptor when it is activated (see, e.g., Table 1), which alter cellular responses. In the case of the PLCγ1-binding motif it is believed that addition of the motif (and possible recruitment of PLCγ1 to the engineered immune receptor) increases stemness or decreases T cell exhaustion, thereby increasing the functionality of the cells in vivo.

In some embodiments the receptor is a chimeric antigen receptor (CAR). In other other embodiments, the receptor may be a chimeric costimulatory receptor (CCR). The structures of CARs, CCRs and other types of immune receptors are described in Sadelain et al (Curr. Opin. Immunol. 2016 41: 68-76).

In some embodiments, the receptor may further comprise: a T cell activation domain. In these embodiments, the receptor may be a chimeric antigen receptor (CAR), where the terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19-4-1BB-CD3ζCAR expressed by lentivirus loaded CTL019 (Tisagenlecleucel-T) CAR-T cells as commercialized by Novartis (Basel, Switzerland).

Binding of the immune receptor to its cognate antigen activates the immune cell. CARs can be designed in several ways (see, generally, e.g., Guedan et al, Methods and Clinical Development 2019 12: 145-156) and may include an extracellular domain that contains an antigen binding domain such as a scFv or nanobody, a hinge, a transmembrane region (which may be derived from CD4, CD8α, or CD28), a costimulatory signaling domains (which may be derived from the intracellular domains of the CD28 family (e.g., CD28 and ICOS) or the tumor necrosis factor receptor (TNFR) family of genes (e.g., 4-1BB, OX40, or CD27), plus one or more of the motifs described above and an ITAM domain, e.g., the signaling domain from the zeta chain of the human CD3 complex (CD3zeta). In practice, any of these domains may be a variation of a wild type sequence. In practice, any of these sequences may be a variant of a wild type sequence, e.g., a sequence that is at least 90%, 95, or 98% identical a sequence described in WO2014127261, for example. Sources for exemplary sequences that can bind to Mesothelin, FAP, Her2, Trop2, GPC3, MUC1, ROR1, EPCAM, ALPPL2, PSMA, PSCA, EGFRviii, EGFR, Claudin18.2, and GD2 are listed above. However, sequences that bind to other antigens are known and/or can be readily made.

As noted above, the immune receptor may be constitutively expressed (in which case its coding sequence will be operably linked to a constitutive promoter, i.e., a promoter that is always “on” in the cell) or induced, e.g., by activation of a proteolytic receptor.

A method of treatment for a cancer associated is also provided. In general terms, this method may comprise administering a cell described above to a subject that has cancer, e.g., solid tumor or a blood cancer. In some embodiments, primary immune cells (e.g., T cells or NK cells, etc.) may be purified from an individual, constructs encoding the above proteins may be introduced into the cells ex vivo, and the recombinant cells may be expanded and administered to the subject, e.g., by injection. Alternatively, allogeneic immune cells may be used.

The antigens to which the immune receptor and BTTS bind depend on which cancer is being treated.

In some embodiments, the T cell activation domain comprises at least one immunoreceptor tyrosine-based activation motif (ITAM). In these embodiments, the ITAM (immunoreceptor tyrosine-based activation motif) may be described by the formula YX₁X₂L/I, where X₁ and X₂ are independently any amino acid. In some cases, the intracellular signaling domain of a subject engineered immune receptor comprises 1, 2, 3, 4, or 5 ITAM motifs. In some cases, an ITAM motif is repeated twice in an intracellular signaling domain, where the first and second instances of the ITAM motif are separated from one another by 6 to 8 amino acids, e.g., (YX₁X₂L/I)(X₃)ₙ(YX₁X₂L/I), where n is an integer from 6 to 8, and each of the 6-8 X₃ can be any amino acid. In some cases, the intracellular signaling domain of a subject engineered immune receptor comprises 3 ITAM motifs. Suitable ITAMs can be derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12; FCER1G (Fc epsilon receptor I gamma chain); CD3D (CD3 delta); CD3E (CD3 epsilon); CD3G (CD3 gamma); CD3Z (CD3 zeta); and CD79A (antigen receptor complex-associated protein alpha chain), although in practice functional variants of these domains (e.g., domains that have at least 90% or 95% sequence identify to a wild type domain) can be used in many cases.

In some embodiments, the engineered immune receptor does not comprise a T cell activation domain. In these embodiments, the receptor may be a chimeric costimulatory receptor (CCR).

In any embodiment, the one or more synthetic co-stimulatory motifs include one or more (e.g., one, two, or at least three) PLCγ1-binding motifs. In these embodiments, the one or more PLCγ1-binding motifs comprise one or more PLCγ1-binding motifs of the consensus sequence (Y)[AFILVWY]×[AFILVWY]. For example, in some embodiments the receptor may comprise one or more of YLVV (SEQ ID NO: 6), YIIP (SEQ ID NO: 2), YLIP (SEQ ID NO: 3), YLRV (SEQ ID NO: 4) or YLVP (SEQ ID NO: 5).

In any embodiment, the one or more synthetic co-stimulatory motifs include one or more (e.g., one, two, or at least three) Tumor necrosis factor receptor-associated factor (TRAF) binding motifs. These TRAF binding motifs may be in addition to one or more PLCγ1-binding motifs, or instead of the one or more PLCγ1-binding motifs. In these embodiments, the one or more synthetic co-stimulatory motifs include one or more motifs of the consensus sequence: (i) Px(Q/E)E, Px(Q/E)xxD or Px(Q/E)xT, where x is any amino acid (which provide a binding site for TRAF1, -2, -3, and -5); (ii) Arg-Leu-X-Ala, where X is be any amino acid and Ala can be replaced by a small uncharged residue (which provides a binding site for TRAF1, -2, -3, and -5); and/or (iii) PxExxZ, where x is any amino acid and Z is acidic or aromatic amino acid (which provides a binding site for TRAF6). For example, of any prior claim, wherein one or more synthetic co-stimulatory motifs include one or more of PVQE (SEQ ID NO: 15), PQQAT (SEQ ID NO: 16) and PQEINF (SEQ ID NO: 17). These motifs are described in Described in Park et al Front Immunol. 2018 9:1999).

As noted above, in some embodiments the one or more synthetic co-stimulatory motifs include one or more TRAF protein binding motifs and one or more PLCγ1-binding motifs. Specifically, the motifs may be used in any combination. For example, a receptor may have a one or two PLCγ1-binding motif as well as one, two or three TRAF (TRAF2, TRAF1 or TRAF6)-binding motifs.

In some embodiments, the receptor may further comprise (i.e., in addition to the one or more synthetic co-stimulatory motifs and other components) a co-stimulatory domain from 4-1BB (CD137), CD28, ICOS, OX-40, BTLA, CD27, CD30, GITR, CD40, CD40L, HVEM, or a TLR, although in practice functional variants of these domains (e.g., domains that have at least 90% or 95% sequence identify to a wild type domain) can be used in many cases.

For example, in any embodiment, a receptor may contain the 4-1BB as well two or three separate binding motifs that bind to PLC and TRAF. See, e.g., SEQ ID NO: 1 below.

In any embodiment, a receptor may have SEQ ID NO: 1 below.

In some embodiments, the extracellular binding domain may comprise the antigen binding domain of a nanobody or scFv, a ligand for a receptor, or a receptor for a ligand. In some embodiments, the extracellular binding domain may recognize tissue-specific or disease specific antigen, e.g., a cancer-associated antigen, where cancer-associated antigens include, e.g., CD19, CD20, CD38, CD30, Her2/neu, ERBB2, CA125, MUC-1, prostate-specific membrane antigen (PSMA), and several others. See, e.g., Dannenfelser (Cell Syst. 2020 11: 215-228), WO2017/193059, WO2020/097395 and PCT/US2021/045796).

In some embodiments, the binding domain of the receptor may be specific for Mesothelin, FAP, Her2, Trop2, GPC3, MUC1, ROR1, EPCAM, ALPPL2, PSMA, PSCA, EGFRviii, EGFR, Claudin18.2, or GD2, for example. In some embodiments, a binding domain of the fusion protein may have HC and LC CDR1, 2 and 3 sequences that are identical to or similar (i.e., may contain up to 5 amino acid substitutions, e.g., up to 1, up to 2, up to 3, up to 4 or up to 5 amino acid substitutions, collectively) to the CDRs of any of the antibodies listed in the publication cited in the table below, which publications are incorporated by reference for those sequences. The framework sequence could be humanized, for example. In some embodiments, the binding domain of the fusion protein may have HC and LC variable regions that are at least 90%, at least 95%, at least 98% or at least 99% identical to a pair of HC and LC sequences listed in the publication cited in the table below, which publications are incorporated by reference for those sequences.

| Antigen binding domain | Exemplary sources of antigen binding sequences |
|---|---|
| Mesothelin (MSLN) | US 2021/0290676, US 2021/0284728 A1, US 2021/0275584 |
| | A1, Feng et al., Mol. Cancer Ther. 8(5): 1113-1118 (2009), US |
| | 2021/0269537 A1, US 2021/0252122 A1, US 2021/0230242 |
| | A1, US 2021/0155702 A1, US 2021/0137977 A1, US |
| | 2021/01016620 A1 |
| FAP | US 2021/0252122 A1, Kakarla et al. Mol Ther. 2013 |
| | August; 21(8): 1611-20, Wang et al. Cancer Immunol Res. 2015 |
| | July; 3(7): 815-826, Petrausch et al. BMC Cancer. 2012; 12: |
| | 615, Tran et al. J Exp Med. 2013 Jun. 3; 210(6): 1125-35. |
| Her2 | US 2021/0299269, US 2021/0290676, US 2021/0137977 A1, |
| | US 2021/01016620 A1, US 2021/0299172 A1 |
| Trop2 | US 2021/0290676, Zhao et al. Am J Cancer Res. 2019; 9(8): |
| | 1846-1856., Bedoya et al. Cytotherapy 2019 May; 21(5): S11- |
| | 12., Sayama et al. Mol Med Rep. 2021 February; 23(2): 92. |
| GPC3 | US 2021/0261646 A1, US 2021/0137977 A1, US |
| | 2021/01016620 A1, Li et al. Am J Transl Res. 2021 Jan. |
| | 15; 13(1): 156-167., Batra et al. Cancer Immunol Res. 2020 |
| | March; 8(3): 309-320. |
| MUC1 | US 2021/0269547 A1, US 2021/0155702 A1, Supimon et al. |
| | Sci Rep. 2021 Mar. 18; 11(1): 6276., Zhou et al. Front Immunol. |
| | 2019 May 24; 10: 1149., Mei et al. Cancer Med. 2020 |
| | January; 9(2): 640-652. |
| ROR1 | US 2021/0290676, Wallstabe et al JCI Insight. 2019 Sep. 19; |
| | 4(18): e126345, US 2021/0137977 A1, Prussak et al. J. Clin. |
| | Oncol. 2020; 38, no. 6_suppl, Srivastava et al. Cancer Cell. |
| | 2021 Feb. 8; 39(2): 193-208.e10. |
| EPCAM | US 2021/0290676, US 2021/0284728 A1, US 2021/0269547 |
| | A1, Qin et al. Oncoimmunology. 2020 Aug. 15; 9(1): 1806009., |
| | Deng et al. BMC Immunol. 2015 Jan. 31; 16(1): 1. |
| ALPPL2 | Su et al Cancer Res. 2020 Oct. 15; 80(20): 4552-4564., |
| | Hyrenius-Wittsten et al. Sci Transl Med. 2021 Apr. |
| | 28; 13(591): eabd8836., WO2017095823A1 |
| PSMA | US 2021/0290676, US 2021/0284728 A1, US 2021/0269547 |
| | A1, US 2021/0252122 A1, US 2021/0137977 A1, US |
| | 2021/0113615 A1 |
| PSCA | US 2021/0290676, US 2021/0269547 A1, Wu et al. Biomark |
| | Res. 2020 Jan. 28; 8: 3., Dorff et al. J. Clin. Oncol. 2020; 38, no. |
| | 6_suppl, US 2020/0308300 |
| EGFRviii | US 2021/0290676, US 2021/0252122 A1, US 2021/0137977 |
| | A1, O'Rourke et al. Sci Transl Med. (2017) 9: eaaa0984, Abbott |
| | et al. Clin Transl Immunology. 2021 May 9; 10(5): e1283. |
| EGFR | US 2021/0290676, US 2021/0269547 A1, US 2021/0155702 |
| | A1, Xia et al. Clin Transl Immunology. 2020 May |
| | 3; 9(5): e01135., Li et al. Cell Death Dis. 2018 February; 9(2): 177., |
| | Liu et al. Clinical Trial Cytotherapy. 2020 October; 22(10): 573-580. |
| Claudin 18.2 | US 2021/0252122 A1, Jiang et al. J Natl Cancer Inst. 2019 Apr. |
| | 1; 111(4): 409-418., Chin et al. J Cancer Res. 2020 April; 32(2): |
| | 263-270., Zhan et al. J. Clin. Oncol. 2019, 37, 2509., Singh et |
| | al. J Hematol Oncol. 2017; 10: 105. |
| GD2 | US 2021/0290676, Seitz et al. Oncoimmunology. 2020; 9(1): |
| | 1683345., Chulanetra et al. Am J Cancer Res. 2020; 10(2): |
| | 674-687., Sujjitjoon et al. Transl Oncol. 2021 |
| | February; 14(2): 100971, Andersch et al. BMC Cancer. 2019 Sep. |
| | 9; 19(1): 895. |

New antigen binding domains may also be generated in the form of immunoglobulin single variable (ISV) domains. The ISV domains may be generated using any suitable method. Suitable methods for the generation and screening of ISVs include without limitation, immunization of dromedaries, immunization of camels, immunization of alpacas, immunization of sharks, yeast surface display, etc. Yeast surface display has been successfully used to generate specific ISVs as shown in McMahon et al. (2018) Nature Structural Molecular Biology 25(3): 289-296 which is specifically incorporated herein by reference.

Immunoglobulin sequences, such as antibodies and antigen binding fragments derived there from (e.g., immunoglobulin single variable domains or ISVs) are used to specifically target the respective antigens disclosed herein. The generation of immunoglobulin single variable domains such as e.g., VHHs or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production.

As noted above, the engineered receptor contains a transmembrane domains (which should be in between the extracellular and intracellular domains). In some embodiments the transmembrane can be the transmembrane domain of a naturally-occurring transmembrane protein. However, this is not necessary because the transmembrane domain can be readily designed using hydrophobic amino acids or a transmembrane domain from another transmembrane protein can be used.

As would be apparent, the fusion protein may have other sequences, e.g., linkers, effector domains, signaling domains, etc., in addition to the domains that are specifically described herein.

A nucleic acid encoding the engineered immune receptor is provided, including vectors and expression cassettes containing the same.

Also provided is an immune cell expressing the engineered immune receptor, wherein binding of the immune receptor to the protein on the surface of the other cell activates the immune cell, where term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

In general terms, if the receptor is a CAR, the cell does not need to be activated by a separate receptor with a costimulatory domain, since the CAR contains both an ITAM and a costimulatory domain. If the receptor is a CCR, then the cell may additional contain a CAR, where the CAR contains an ITAM and may or may not contain a costimulatory domain. In these embodiments, binding of the CCR to a ligand on another cell enhances immune cell activation by the CAR. In some embodiments, the extracellular part of the CCR may bind to a cancer antigen (e.g., a different cancer antigen to the CAR) but in other embodiments, the extracellular part of the CCR may bind to the ligands for PD1, CTLA4, BTLA, CD160, KRLG-1, 2B4, Lag-3, or Tim-3, etc. thereby providing a way to turn a negative signal into a positive one. CCRs can also be dimerized with CARs using a dimerizing agent, e.g., FK506 or the like.

The cell can be a primary T cell in some cases, where the term “T cell” includes all types of immune cells expressing CD3 including T-helper cells (CD4⁺ cells), cytotoxic T-cells (CD8⁺ cells), T-regulatory cells (Treg) and gamma-delta T cells. A “cytotoxic cell” includes CD8⁺ T cells, natural-killer (NK) cells, and neutrophils, which cells are capable of mediating cytotoxicity responses and are of particular interest. In some embodiments, the immune may be a myeloid or lymphoid cell.

Also provided is a method for killing cells. These embodiments may comprises introducing an immune cell as described above with a target cell that is protein on its surface that is recognized by the engineered immune receptor. In these embodiments, the immune cell is activated by binding of the engineered immune receptor to the protein on the other cell, and the immune cell kills the target cell. The target cell can be a cancer cell, for example. This method may be practiced in vitro, ex vivo or in vivo. For example, the method may be used to treat a subject for a disease, e.g., cancer, where the method comprises administering to the subject the immune cell.

### Embodiments

Embodiment 1. An engineered immune receptor (e.g., a chimeric antigen receptor (CAR or chimeric costimulatory receptor (CCR)) comprising: (a) an extracellular binding domain that binds to a protein on the surface of another cell, e.g., a nanobody, scFv, ligand for a receptor, or receptor for a ligand; (b) a transmembrane domain; and (c) a motif that binds to a protein listed in Table 1, (e.g., a PLCγ1-binding motif) or any combination thereof; wherein the engineered immune receptor comprises a T cell activation domain, e.g., an ITAM, if the receptor is a CAR and wherein the engineered immune receptor does not comprise a T cell activation domain if the receptor is a CCR.

Embodiment 2. The engineered immune receptor of embodiment 1, wherein motif of (c) is a PLCγ1-binding motif of the consensus sequence (Y)[AFILVWY]x[AFILVWY].

Embodiment 3. The engineered immune receptor of embodiment 1 or 2, wherein motif of (c) is YLVV (SEQ ID NO: 6), YIIP (SEQ ID NO: 2), YLIP (SEQ ID NO: 3), YLRV (SEQ ID NO: 4) or YLVP (SEQ ID NO: 5).

Embodiment 4. The engineered immune receptor of any prior embodiment, wherein the receptor is a CAR and the ITAM is from DAP12; FCER1G (Fc epsilon receptor I gamma chain); CD3D (CD3 delta); CD3E (CD3 epsilon); CD3G (CD3 gamma); CD3Z (CD3 zeta); or CD79A (antigen receptor complex-associated protein alpha chain), or a variant thereof.

Embodiment 5. A nucleic acid encoding the engineered immune receptor of any of embodiments 1-4.

Embodiment 6. An immune cell expressing the engineered immune receptor of any of embodiments 1-4, wherein binding of the engineered immune receptor to the protein on the surface of the other cell activates the immune cell.

Embodiment 7. The immune cell of embodiment 5, wherein the cell is a myeloid or lymphoid cell.

Embodiment 8. The immune cell of embodiment 6, wherein the lymphoid cell a T lymphocyte, a B lymphocyte or a Natural Killer cell.

Embodiment 9. A method of treating a subject for a disease, the method comprising: administering to the subject a cell of any of embodiments 6-8.

Embodiment 10. The method of embodiment 9, wherein the disease is cancer.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

In these examples, it is shown that chimeric antigen receptor (CAR) costimulatory domains steer the phenotypic output of therapeutic T cells. In most cases these domains are derived from native immune receptors, composed of signaling motif combinations selected by evolution. To explore if non-natural combinations of signaling motifs could drive novel cell fates of interest, a library of CARs containing ˜2,300 synthetic costimulatory domains was built from combinations of 13 peptide signaling motifs. The library produced CARs driving diverse fate outputs, which were sensitive to motif combinations and configurations. Neural networks trained to decode the combinatorial grammar of CAR signaling motifs allowed extraction of key design rules. For example, the non-native combination of TRAF- and PLCγ1-binding motifs was found to simultaneously enhance cytotoxicity and sternness, a clinically desirable phenotype associated with effective and durable tumor killing. The neural network accurately predicts that addition of PLCγ1-binding motifs improves this phenotype when combined with TRAF-binding motifs, but not when combined with other immune signaling motifs (e.g. PI3K- or Grb2-binding motifs). This work shows how libraries built from the minimal building blocks of signaling, combined with machine learning, can efficiently guide engineering of receptors with desired phenotypes.

Costimulatory domain engineering has reportedly been limited to the addition of intact natural domains such as 4-1BB, CD28, or the IL2Rb chain, effectively using naturally occurring signaling sentences (motif combinations). In the present stud motifs from receptors were used as words to generate thousands of novel signaling sentences that drive T cells to distinct cell fates, potentially yielding more diverse and nuanced phenotypic meaning. Augmenting experimental analysis of a subset of receptors with neural network analysis allows one to explore a much larger region of this combinatorial motif space. In particular, the non-natural combination of TRAF- and PLCγ1-binding motifs may be useful in CAR T cell therapies.

### Methods

Viral vector construction: Codon-optimized DNAs encoding the variable library parts were codon optimized for expression in human cells using ThermoFisher GeneArt's website tool and synthesized by ThermoFisher GeneArt. A pHR lentiviral vector containing an SFFV promoter followed by DNA encoding the aCD19 scFv and the CD8a hinge and transmembrane domain was BamHI restriction digested. DNA encoding a BamHI cut site followed by CD3ζ-P2A-EGFP was subcloned into the digested pHR-aCD19-(BamHI)-CD3ζ-P2A-EGFP vector to create library backbone. DNA encoding library parts, as well as DNA encoding 4-1BB, CD28, and ICOS, was subcloned into the library backbone and the cloning product was again BamHI digested. This was repeated to create vectors with 3 variable library parts. All constructs were built via in-fusion cloning (Clontech #ST0345) and sequence verified before use. Amino acid sequences for library parts were as follows. P1: DYHNPGYLVVLPDSTP (SEQ ID NO: 20), P2: EELDENYVPMNPNSPP (SEQ ID NO: 26), P3: EEGAPDYENLQELNHP (SEQ ID NO: 27), P4: LGSNQEEAYVTMSSFYQNQ (SEQ ID NO: 28), P5: LPMDTEVYESPFADPEEIR (SEQ ID NO: 30), P6: KPMAESITYAAVARHSAG (SEQ ID NO: 31), P7: LPTWSTPVQPMALIVLG (SEQ ID NO: 21), P8: PAPSIDRSTKPPLDRSL (SEQ ID NO: 29), P9: GSNTAAPVQETLHGCQ (SEQ ID NO: 22), P10: DDSLPHPQQATDDSGHES (SEQ ID NO: 23), P11: KAPHAKQEPQEINFPDDLP (SEQ ID NO: 24), P12: GSGPGSRPTAVEGLALGSS (SEQ ID NO: 25), P13: SAGSAGSAGSAGSAGSAG (SEQ ID NO: 19).

Cell lines: Nalm 6 cell lines were originally obtained from ATCC (CRL-3273) and were stably transduced with mCherry and firefly luciferase. Nalm 6 cell lines were cultured in RPMI-1640+GlutaMAX (Gibco #72400-047) supplemented with 10% FBS (UCSF Cell Culture Facility).

Primary human T cell isolation and culture: Primary CD4+ and CD8+ T cells were isolated from blood of anonymous donors by negative selection using the Human CD4+ T cell isolation kit and Human CD8+ T cell isolation kit (STEMCELL Technologies #17952 and #17953). T cells were cryopreserved in RPMI1640 (UCSF cell culture core) with 20% human AB serum (Valley Biomedical, #HP1022HI) and 10% DMSO. Upon thawing, T cells were cultured in human T cell medium (HTCM) consisting of X-VIVO 15 (Lonza #04-418Q), 5% Human AB serum, 1 mM 2-mercaptoethanol (Gibco #21985-023), and 10 mM neutralized N-acetyl L-Cysteine (Sigma-Aldrich #A9165) supplemented with 30 units/mL IL-2 (NCI BRB Preclinical Repository). Before co-culture with Nalm 6, T cells were transferred to HTCM without IL-2.

Lentiviral Transduction and Sorting of Human T Cells: Pantropic vesicular stomatitis virus G pseudotyped lentivirus was produced via transfection of LentiX 293T cells (Clontech #11131D) with a pHR′ SIN:CSW transgene expression vector and the viral packaging plasmids pCMVdR8.91 and pMD2.G using FuGENE HD (Promega, #E2312). Primary T cells were thawed the same day and after 24 h in culture, were stimulated with Dynabeads Human T-Activator CD3/CD28 (Life Technologies #11131D) at 25 μL per 1×10⁶ T cells. At 48 h (day 2), viral supernatant was harvested via centrifugation at 500 G for 5 min, and the primary T cells were exposed to the virus for 24 h in a 6-well plate (pooled screens) or in 96-well plates (arrayed screens). Dynabeads were removed at day 5 post-T cell stimulation. For pooled screens, GFP+ T cells were sorted on day 6 post-T cell stimulation with a FACSAria II. Assays were performed 10 days after removal of Dynabeads.

Arrayed Screening: CARs were constructed as described above in Viral Vector Construction. An additional pooled CAR sub-library was constructed with enriched concentration of DNA corresponding to P1, P4, P7, P9, and P10 on the basis of their high proliferation, degranulation, and memory formation in the pooled screening assay. Pooled CAR library DNA was used to transform 5-alpha F′ I^{q} competent E. coli cells (New England BioLabs C2992H), which were then plated on LB/Carbenicillin. At 24 hours 384 colonies (288 from the unbiased library, and 96 from the high-performance sub-library) were picked and miniprepped, added to 96-well plates and sequence verified. Wells with failed sequencing results or unidentifiable sequences were removed from plates and the well contents were replaced with duplicates of nearby wells, TE buffer (for empty well controls), or standard costimulatory domain (4-1BB, CD28) controls. CARs containing 4-1BB, CD28, and ICOS costimulatory domains and P1-P13 were left in place, but excluded from the analysis.

Primary human T cells transduced with CAR library constructs were mixed with Nalm 6 to reach 1×10⁶ T cells per mL and 2×10⁶ per mL Nalm 6 and centrifuged at 300 g for 2 min. For day 3, 5, and 7 challenges with Nalm 6, 80 μL of co-cultured T cells and Nalm 6 were added to 120 μL of Nalm 6 at 2×10⁶ per mL and centrifuged at 300 g for 2 min.

For analysis of cell surface receptor expression, samples were centrifuged at 500×g for 5 min and resuspended in a 50 μL volume with the appropriate antibodies diluted 1:50 in calcium-free magnesium-free PBS with 5% FBS and 5 mM EDTA. After a 30-min incubation at room temperature, samples were washed twice calcium-free magnesium-free PBS with 5% FBS and 5 mM EDTA. (FBS; UCSF Cell Culture Facility). Samples were analyzed for protein expression on a BD LSRII. Antibodies are as follows: APC Mouse anti-human KLRG1 clone SA231A2 (BioLegend #367716), BV421 Mouse anti-human IL7Ra clone HIL-7R-M21 (BD Biosciences #562436), BV786 Mouse anti-human CD62L clone SK11 (BD Biosciences #565311), AF700 Mouse anti-human CD45RA clone HI100 (BD Biosciences #560673), PE Mouse anti-human CD4 clone RPA-T4 (BD Biosciences #555347).

Pooled Screening: Primary human T cells transduced with pooled virus for the CAR library were mixed with Nalm 6 to reach 1×10⁶ T cells per mL and 2×10⁶ per mL Nalm 6. For day 3, 5, and 7 challenges with Nalm 6, co-cultured T cells and Nalm 6 were centrifuged at 400 g for 4 min and resuspended at 1×10⁶ per mL in ⅓ current HTCM and ⅔ fresh HTCM. Additional Nalm 6 were added at 2×10⁶ per mL.

For extracellular staining, samples were centrifuged at 500 g for 5 minutes and resuspended in FACS buffer with 1:50 PE anti-human CD4 antibody, 1:50 BV421 mouse anti-human CD45RA antibody, and 1:50 AF647 mouse anti-human CCR7 antibody. After a 30-min incubation at room temperature, samples were washed twice, and resuspended in FACS buffer. Samples were sorted on a BD FACS AriaII.

For analysis of degranulation on Day 9 Nalm 6 challenge, samples of 1×10⁶ per mL pooled CAR T cells with 2×10⁶ per mL Nalm 6 were centrifuged at 300×g for 2 min in 96-well flat-bottom plates and incubated in HTCM with 1× Brefeldin-A/GolgiPlug (BDBiosciences #555029), 1× Monensin/GolgiStop (BD Biosciences #554724), and 1:50 APC anti-human CD107A antibody at 37 C and 5% C02 for 5 hours. At 5 hours, samples were centrifuged at 300 g for 2 minutes and supernatant was removed. Samples were resuspended in FACS buffer with 1× GolgiPlug, 1× GolgiStop, and 1:50 PE anti-human CD4 antibody. After a 30-min incubation at room temperature, samples were washed twice, and resuspended in FACS buffer. Samples were sorted on a BD FACS AriaII. Antibodies were as follows: PE Mouse anti-human CD4 clone RPA-T4 (BD Biosciences #555347), BV421 Mouse anti-human CD45RA clone HI100 (BD Biosciences #562885), AF647 Mouse anti-human CCR7 clone 150503 (BD Biosciences #560816), APC Mouse anti-human CD107A clone H4A3 (Biosciences #641581).

Genomic DNA was extracted from sorted T cells using the Macherey-Nagel NucleoSpin Tissue XS kit (Takara #740901.250). DNA encoding the CAR costimulatory domain was amplified from the extracted genomic DNA using the forward primer 5′-TCGTCGGCAGCGTCAGATGTGTATAAGAGACAGNNNNNACTGGTTATCACCCTTTA CTGC-3′ (SEQ ID NO: 32) (Integrated DNA Technologies) and reverse primer 5′-GTCTCGTGGGCTCGGAGATGTGTATAAGAGACAGNNNNNCTTGTAGGCGGGAGCAT -3′ (SEQ ID NO: 33) (Integrated DNA Technologies). Indexes were added to the amplified DNA using i5 and i7 primers from the Nextera XT Index Kit (Illumina #FC-131-1002). Indexed samples were loaded into a MiSeq Reagent Kit v3 600-cycle (Illumina #MS-102-3003) cartridge sequenced on a MiSeq (Illumina). Reads for each CAR costimulatory domain construct were counted using software provided by Ian Webster at Zenysis Technologies.

Assessment of Akt, ERK1/2, and NFkB phosphorylation: For intracellular phospho-signaling analysis, T cells and Nalm 6 cells were plated at 1:2 ratio in HTCM and centrifuged at 400 g for 3 minutes. Cells were plated and centrifuged at t=0, 30, 45, 50, 55, and 59 min to obtain timepoints for approximately 60, 30, 15, 10, 5, and 1 minutes of T cell:Nalm 6 engagement. After the final sample was centrifuged, all samples were mixed 1:1 with prewarmed CytoFix Fixation Buffer (BDBiosciences #554655) and incubated at room temperature for 15 minutes. Samples were washed twice with FACS buffer, vigorously vortexed, and permeabilized with BD Phosflow Perm Buffer II (BDBiosciences #558050) by incubating overnight at −20 C. Permeabilized samples were washed twice with FACS buffer and resuspended in 50 mL of FACS buffer with 1:50 anti-human phospho-NFkB antibody, 1:50 anti-human phospho-Akt antibody, and 1:50 anti-human phospho-ERK1/2 antibody. After a 60-minute incubation, samples were washed twice with FACS buffer and analyzed by flow cytometry on a BD LSRII. Antibodies are as follows: BV421 Mouse anti-human NFkB pS529 clone K10-895.12.50 (BD Biosciences #565446), PerCP-Cy5.5 Mouse anti-human ERK1/2 pT202/pY204 clone 20A (BD Biosciences #560115), PE-CF594 Mouse anti-human Akt pS473 clone M89-61 (BD Biosciences #562465), AF647 Mouse anti-human Akt pS473 clone M89-61 (BD Biosciences #560343).

Flow cytometry data were analyzed in FlowJo (BD) software to calculate mean fluorescence intensity (MFI) for pErk1/2, pNFkB, and pAkt channels. MFI kinetic traces were interpolated and integrated in Mathematica (Wolfram) to calculate the total change over 60 minutes in MFI for CAR T cell samples relative to the change in MFI of the untransduced control. Integrated changes in MFI were normalized to 4-1BB measurements to standardize experiments performed on different days.

Mice: All mouse experimental procedures were conducted according to Institutional Animal Care and Use Committee (IACUC)-approved protocols. Female immunocompromised NOD-SCID-Il2rg^{-/-} (NSG) mice were obtained from UCSF breeding core. On Day 1, mice were inoculated with 0.5×10⁶ Nalm 6 leukemia via tail vein injection. On Day 4, mice were injected with 3×10⁶ T cells via tail vein injection. Leukemia progression was measured by bioluminescent imaging using the IVIS 100 (Xenogen) preclinical imaging system. Images were acquired 15 minutes following intraperitoneal (i.p.) injection with 150 mg/kg of D-luciferin (Gold Technology #LUCK-100). Display and adjustment of bioluminescence intensities was performed using the Living image 4.5.4 software (Perkin Elmer). Mice were humanely euthanized when IACUC-approved endpoint (hunching, neurological impairments such as circling, ataxia, paralysis, limping, head tilt, balance problems, seizures, tumor volume burden) was reached (10 mice per group).

Data Preparation: For the arrayed data, in addition to the positional information of the combinational motifs, the initial CAR T cell number is a variable which affects the experimental output. Both are inputs of the machine learning algorithms. D2 was randomly split, 90% for training and 10% for test, (we repeated this splitting process until duplicate motif combinations were found either exclusively in the training sets or exclusively in the test sets) ensuring all the motif combinations in the test data are different from those in the training.

One-hot encoding was used to input motif combinations into the machine learning algorithms. Each motif position was described by a vector of fifteen 0s, and one 0 in each vector was replaced with a 1 corresponding to the absence of a motif (replace the first 0 with 1), the presence of a motif (replace the 0 equal to the part number+1 with 1), or the presence of CD3ζ (replace the 15th 0 with 1). Up to 5 motif positions were allowed, as well as CD3ζ, for a total of 6 vectors. This allows for inclusion of a small number of CARs that contained more than 3 motifs, and allows flexible inclusion of additional data for CARs with more than 3 motifs.

Machine learning Framework: In this work, a Convolutional Neural Networks (CNN) was used, followed by a Long Short-Term Memory (LSTM) network together with fully connected layers. The code is implemented in either Mathematica (Wolfram) or Python 3.7.8 and TensorFlow v2.4.1, both of which produce nearly identical results. The Mathematica analysis is described below and more detailed analysis is described in the forthcoming companion paper. The neural network uses the A×B matrices as inputs and outputs one value corresponding to one of the phenotypes (cytotoxicity and sternness). Between the input and output layers, there are two convolutional layers, 1 LSTM layer, 1 dropout layer, and several fully connected layers. The convolutional layers detect spatial correlations in input data and the LSTM layer learns the long-term dependencies of the sequence data. Dropout regularization was used to prevent over-fitting. The dropout layer connects to fully connected layers which are then flattened and catenated with the cell number input and connect to a dense layer. Linear activation function was used to connect this dense layer and the final output layer. For training in Mathematica, mean squared error loss and ADAM optimization algorithm with automatic learning rate were used, and training was over 200 iterations.

The methods were compared with other widely used machine learning regression methods, such as k-nearest neighbor regression, linear regression, nearest neighbors, random forest regression, and gradient boosted regression. The method has the best performance and predictive power compared to other methods. Details of the analysis can be found in the forthcoming companion paper.

Selection of Neural Network Hyperparameters

The hyperparameters for layers in the neural networks were tuned to find optimal hyperparameters for the cytotoxicity and stemness datasets. The tuned hyperparameters include convolutional layers filters (10, 20, 50), kernel size (2, 3, 4, 5); LSTM layer units (2, 4, 8), dropout layer dropout rate (0, 0.1, 0.2), and fully connected layer units (6, 14, 64).

Hyperparameters were tuned as follows: a grid search of hyperparameters was performed and each parameter set was scored by 10-fold cross validation of the training set. The best-performing 10 hyperparameter sets for each dataset (cytotoxicity or stemness) were selected using the K-fold averaging cross validation (ACV) method and used to train 10 neural networks whose outputs were then averaged(26). The trained neural networks were used to simulate the cytotoxicity and sternness for the 2379 combinations of 1, 2, or 3 variable motifs at a fixed initial cell count of 2000 cells (corresponding to 2000 CAR T cells in 40 mL of flow cytometry sample).

Hyperparameters for final neural networks are available in tables shown below:

TABLE 2
Hyperparameters for Neural Networks - Cytotoxicity Data

| Neural | | Convolutional | LSTM | | Fully |
|---|---|---|---|---|---|
| Network | Convolutional | Layer | Layer | Dropout Layer | Connected |
| Model | Layer Filters | Kernel Size | Units | Dropout Rate | Layer Units |
| NN2: | 10 | 2 | 8 | 0.01 | 16 |
| NN3: | 20 | 4 | 8 | 0.2 | 4 |
| NN4: | 10 | 2 | 4 | 0.01 | 64 |
| NN5: | 20 | 4 | 2 | 0.01 | 16 |
| NN6: | 20 | 2 | 4 | 0.01 | 4 |
| NN7: | 10 | 5 | 4 | 0.01 | 16 |
| NN8: | 20 | 5 | 4 | 0.01 | 4 |
| NN9: | 20 | 2 | 4 | 0.01 | 4 |
| NN10: | 50 | 5 | 4 | 0.01 | 4 |

TABLE 3
Hyperparameters for Neural Networks - Stemness Data

| Neural | | Convolutional | LSTM | | Fully |
|---|---|---|---|---|---|
| Network | Convolutional | Layer | Layer | Dropout Layer | Connected |
| Model | Layer Filters | Kernel Size | Units | Dropout Rate | Layer Units |
| NN1: | 20 | 4 | 2 | 0.01 | 64 |
| NN2: | 20 | 4 | 2 | 0.1 | 4 |
| NN3: | 10 | 2 | 8 | 0.01 | 64 |
| NN4: | 10 | 4 | 2 | 0.01 | 64 |
| NN5: | 10 | 5 | 8 | 0.01 | 16 |
| NN6: | 10 | 3 | 8 | 0.1 | 64 |
| NN7: | 10 | 2 | 2 | 0.01 | 16 |
| NN8: | 50 | 4 | 4 | 0.2 | 4 |
| NN9: | 10 | 5 | 8 | 0.01 | 16 |
| NN10: | 20 | 2 | 4 | 0.01 | 4 |

Ensemble Method

Due to the stochastic nature of network initialization and dropout, as well as the availability of a limited training set, every neural network is unique in terms of the parameterization of the network connections(27, 28). To mitigate the potential impact of this issue, an ensemble decision method was implemented to obtain consensus prediction from ten identical neural networks. Details can be found in the forthcoming companion paper.

Distribution analysis: Distribution analysis was performed in Mathematica (Wolfram). CARs were sorted by proliferation (lowest enrichment to highest enrichment), cytotoxicity (highest Nalm 6 survival to lowest Nalm 6 survival), or stemness (lowest % IL7Ra+/KLRG1− to highest % IL7Ra+/KLRG1−) and assigned percentiles from 0 to 100. Individual parts or motif analysis was performed by selecting all CARs that contain a given part of interest. Pairs of parts or motifs analysis was performed by selecting all CARs that contain a given pair of parts. Position analysis was performed by selecting all CARs that contain a given part at a position of interest. Distributions for selected CARs were constructed using the HistogramDistribution functionality and smoothed by using the PDF (probability distribution function) functionality to calculate the probability from 2.5^{th} percentile to 97.5^{th} percentile in steps of 5. The mean and standard error of the mean for each distribution was calculated by repeating the above processing for each of 10 neural networks (for predicted array screen data) or for experimental replicates (pooled screen data).

### Results

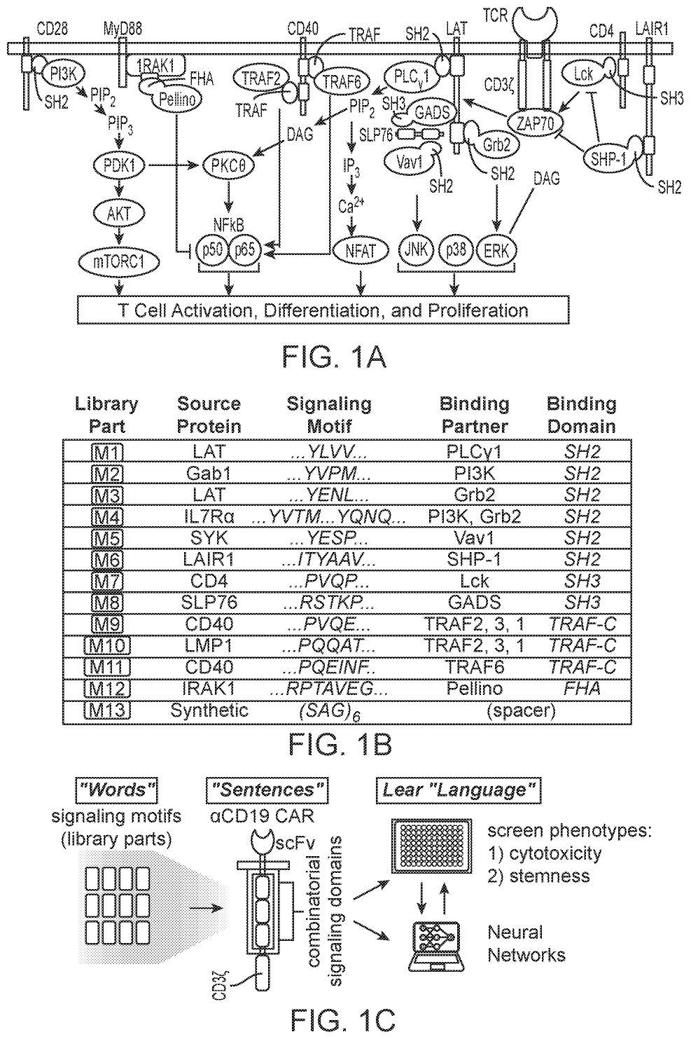

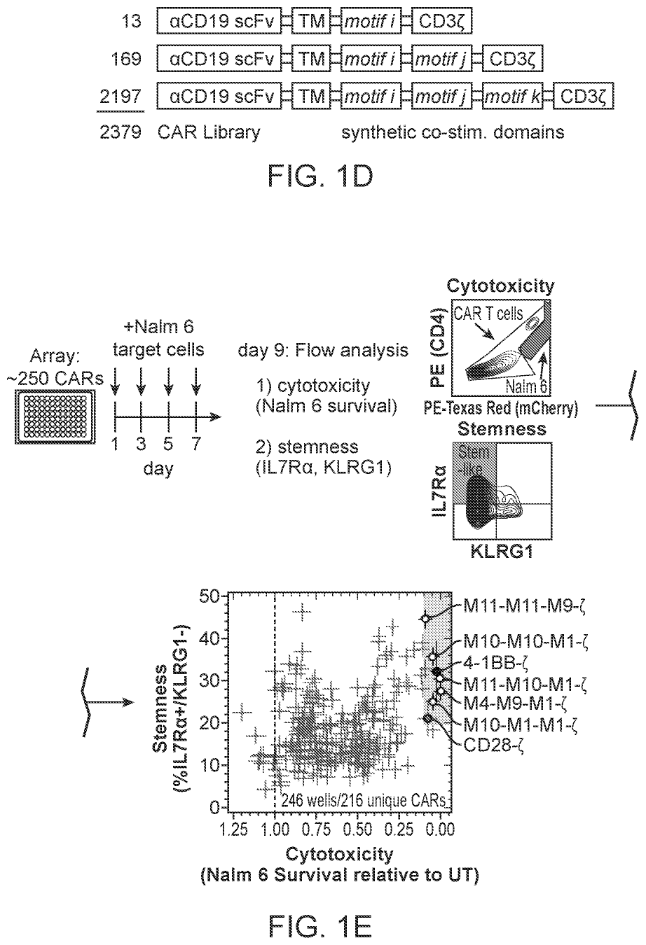

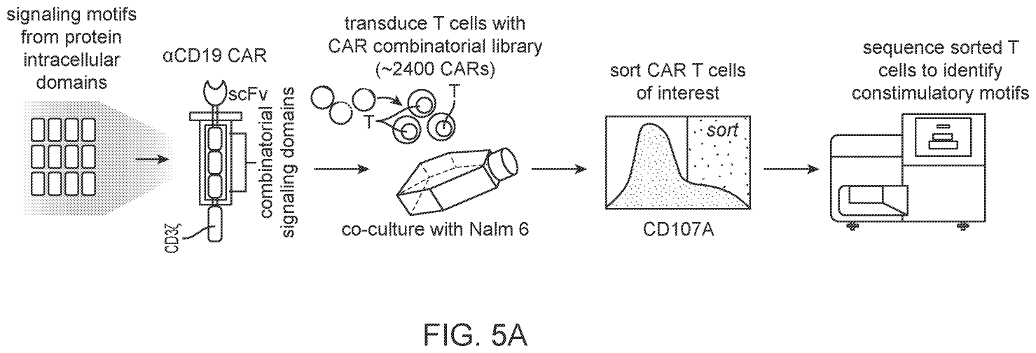

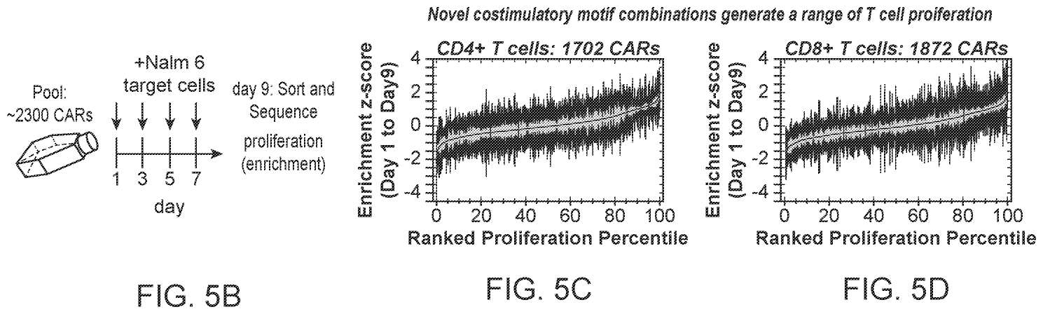

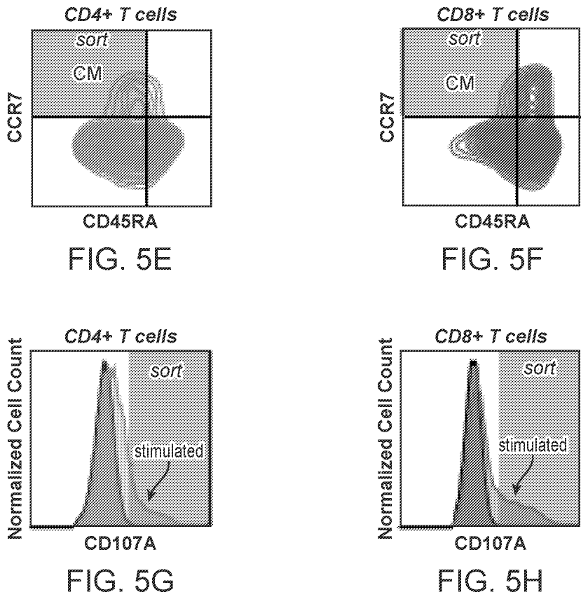

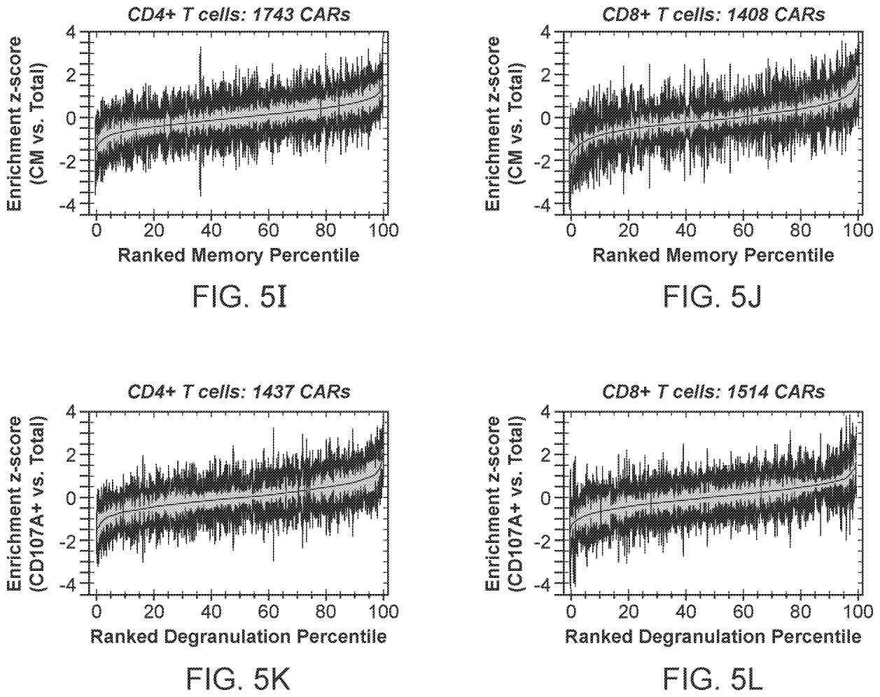

To construct a combinatorial library of CAR signaling domains, the Eukaryotic Linear Motif Database (ELM)(17) and primary literature was searched and a collection of 12 peptide motifs from natural signaling proteins known to recruit key downstream signaling proteins involved in T cell activation was created. The motifs in the library recruit proteins such as PLCγ1, TRAF1/2/3/5, TRAF6, Grb2, GADS, SHP-1, Vav1, PI3K, Lck, and Pellino protein. For example library motif 1 is derived from LAT and contains the core motif YLVV(SEQ ID NO: 6)—which binds the N-terminal SH2 domain of PLCγ1 with high specificity(18). Motif 6, contains the motif ITYAAV (SEQ ID NO: 12) from the protein LAIR1, which binds the phosphate SHP-1 via its SH-2 domain (19). In addition to the 12 signaling motifs, a spacer motif was included as the 13^{th} component in the library. The combinatorial library was constructed within the context of an anti-CD19 CAR (containing an anti-CD19 extracellular scFv and a CD3ζ signaling domain). The synthetic costimulatory domains had either one, two, or three signaling motifs. The 13 motifs were randomly inserted in positions i, j, and k to yield 2379 unique motif combinations (FIG. 1A-D). To confirm that the library displayed sufficient phenotypic diversity, low resolution pooled screens were performed, in which primary human T cells were transduced at low MOI and the pool with Nalm 6 leukemia cells (CD19+) was activated for 8-9 days. Using FACS-based sequencing enrichment assays, a diverse range of phenotypic outputs for T cell proliferation, central memory formation (CD45RA+/CD62L− T cells), and degranulation (CD107A+ T cells, a proxy for cytotoxic response) (FIG. 5) were observed.

To then screen the library at higher resolution, bacteria were transformed with library plasmid stocks and randomly picked colonies to select a subset of over 200 novel CARs from the combinatorial library to characterize in an arrayed screen (FIG. 1F). An arrayed screen was important because immune paracrine signaling could confound analysis of mixed CAR T cell populations and allow the emergence of “cheater” individuals. CAR T cells in the arrayed screen were activated by co-culturing with Nalm 6 cells for 8-9 days. Four pulses of Nalm 6 cells were used to mimic longer term stimulation that can exacerbate T cell exhaustion. At the end of the co-culture, flow cytometry was performed to assess cytotoxicity (Nalm 6 cell killing), and maintenance of T cell populations with markers of central memory/naïve state (CD45RA and CD62L) and stemness (IL7Ra and KLRG1) (20, 21).

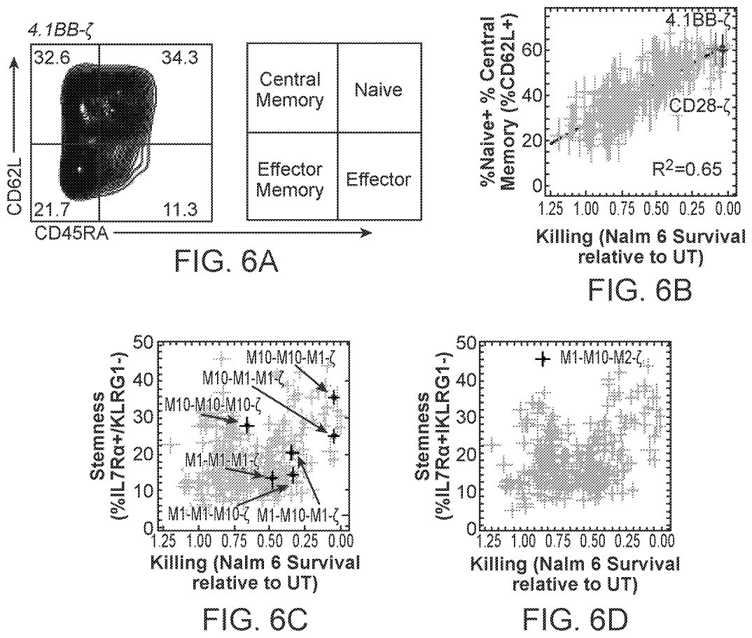

The CARs in the arrayed screen displayed a range of cytotoxicity as well as sternness. The total naïve and central memory population was positively correlated with cytotoxicity (FIG. 6). However, cytotoxicity and sternness were uncoupled. This observation underscores the ability of novel costimulatory domains to drive CAR T cells to varied cell fates with unique combinations of phenotypes. Several novel costimulatory domains produced cytotoxicity and stemness comparable to 4-1BB. Many of these contained motifs that recruit both TRAFs (motif 9, motif 10, motif 11) and PLCγ1 (motif 1). For example M10-M10-M1-z, M-10-M1-z, M11-M10-M1-z, and M4-M9-M1-z all showed robust cytotoxicity and sternness.

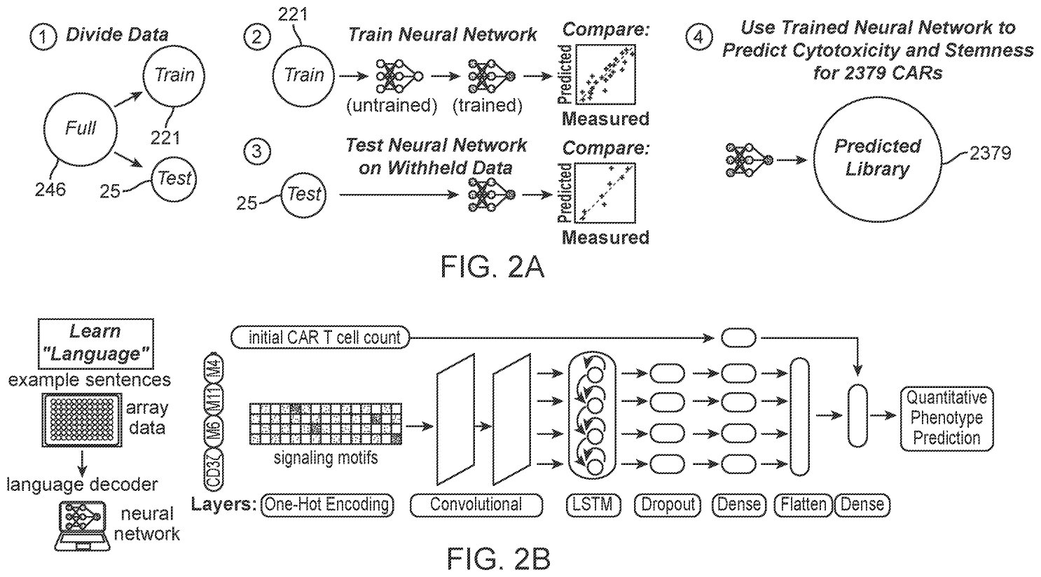

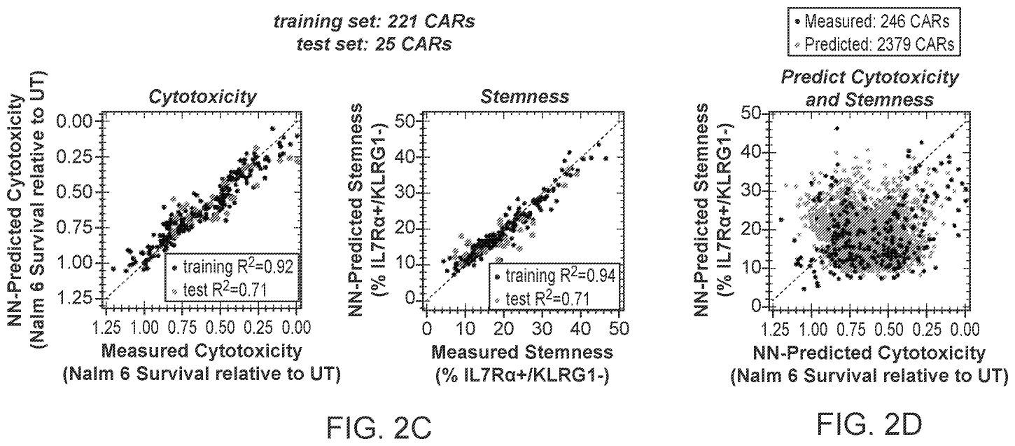

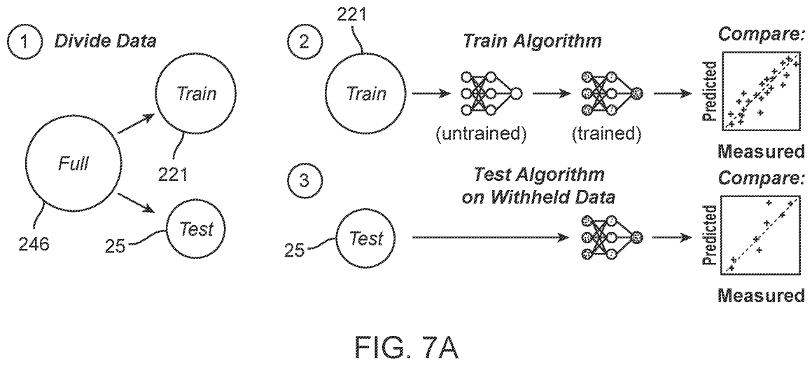

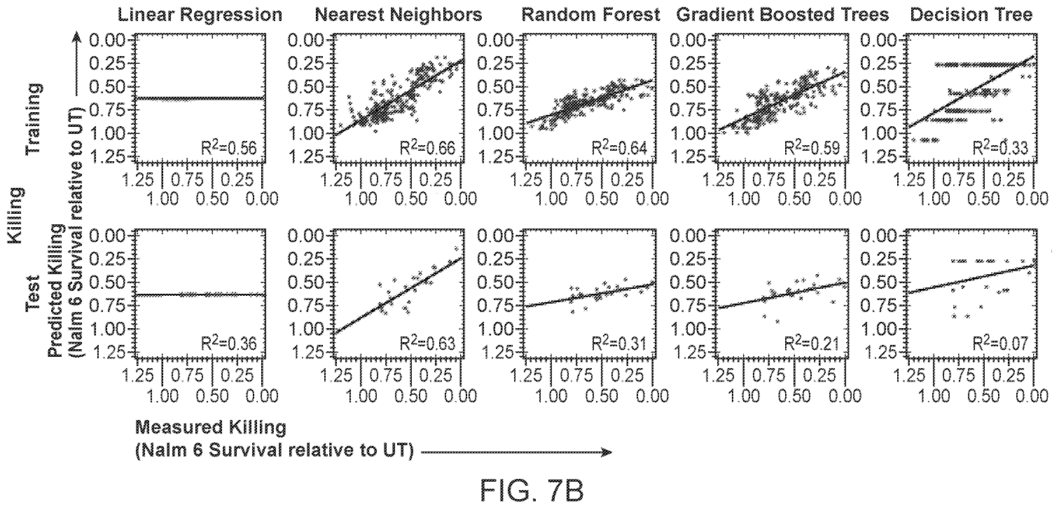

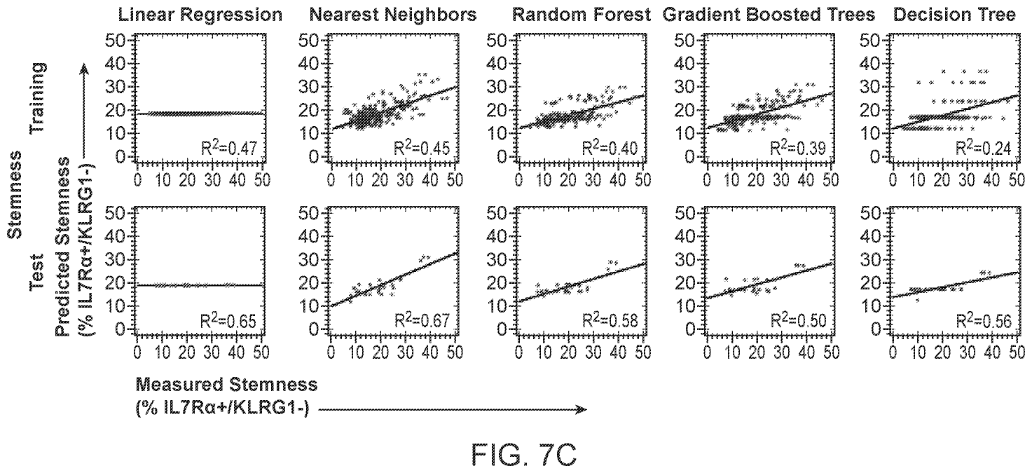

The diverse cytotoxicity and sternness profiles observed in this arrayed screen suggest a complex relationship between signaling motif combinations and arrangement, and resulting T cell phenotypes. To leverage the combinatorial nature of the costimulatory domain library, machine learning was used to decode the “language” of signaling motifs that relates motif combinations to cytotoxicity and sternness outputs. The arrayed screen data was separated into a training sets (221 examples) and a test set (25 examples). These data sets were then used to train several machine learning algorithms to predict cytotoxicity and stemness based on costimulatory domain identity and arrangement (FIGS. 7, 2A). Neural networks (FIG. 2B) were best able to recapitulate the measured phenotypic in the training data (FIG. 2C) and to effectively predict the phenotypes in the test set (FIG. 2D). For both cytotoxicity and stemness training and test sets, the neural network was able to capture much of the relationship between signaling motif composition and phenotype, with R² values of approximately 0.7-0.9.

The trained neural networks then allowed one to predict the CAR T cell cytotoxicity and sternness that would result from each of the 2379 motif combinations in the 1-3 part combinatorial library (FIG. 2D), including those that were not part of the smaller arrayed screen. These simulated 2379 CARs sample the entire combinatorial space of the library, providing a dataset from which design rules could be extracted. Three types of analysis are described below: 1) the overall contribution of each individual motif to a particular phenotype (without regard to combinatorial context); 2) identification of pairwise motif combinations that promote particular phenotypes, and 3) positional dependence of motifs.

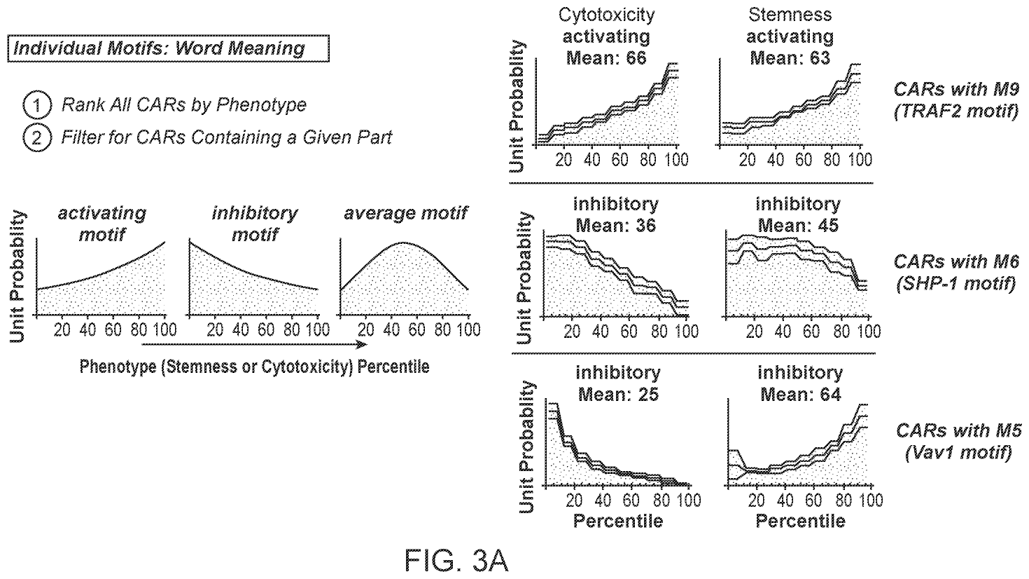

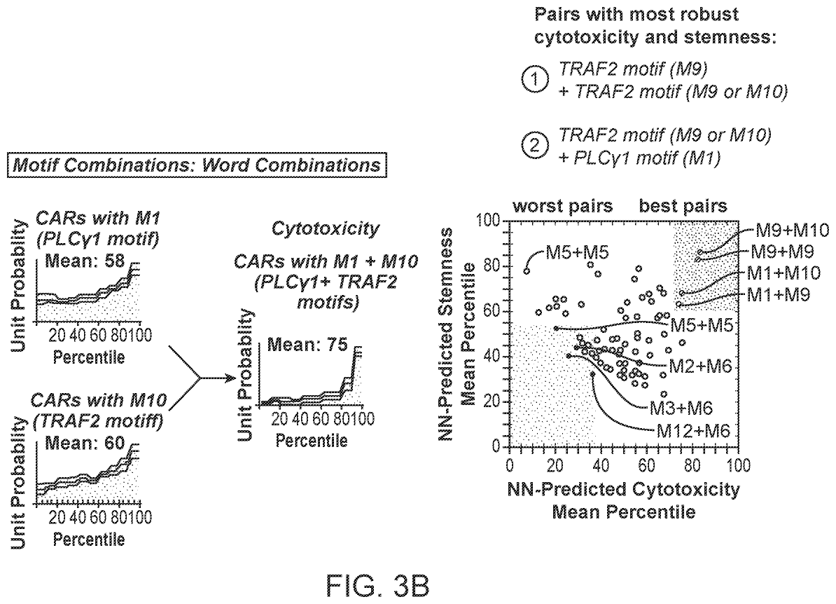

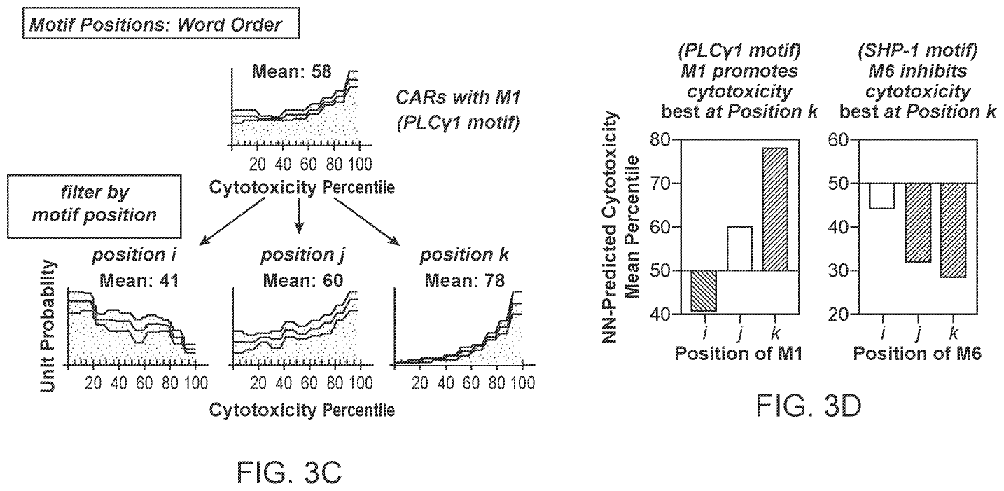

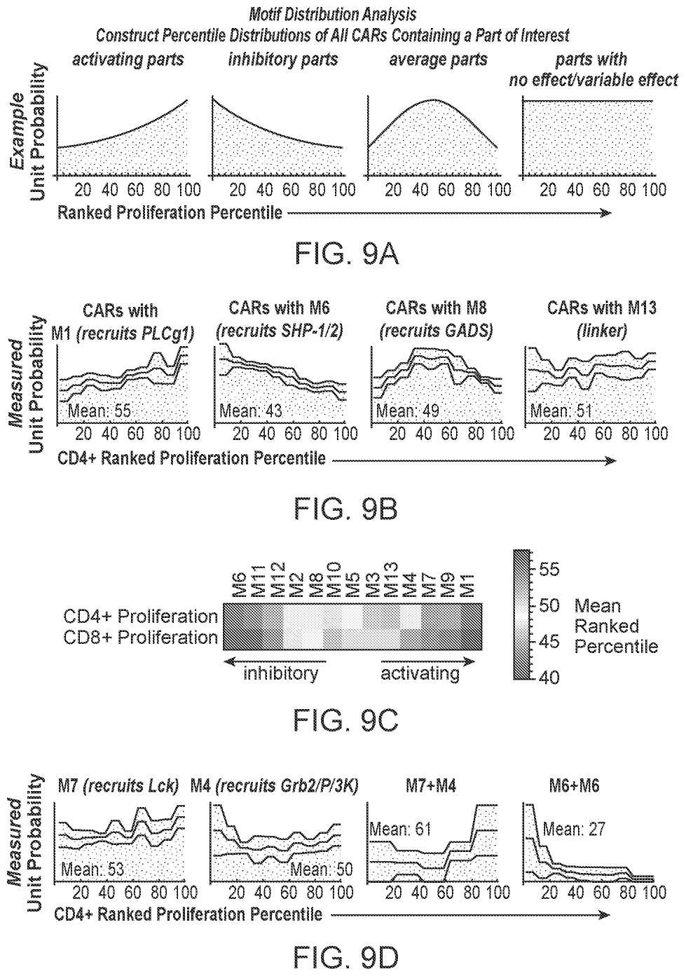

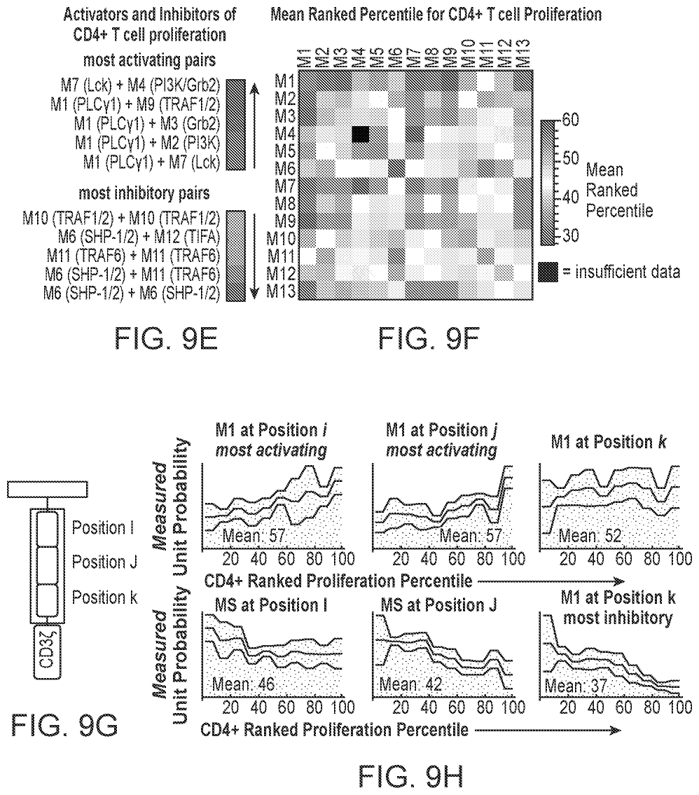

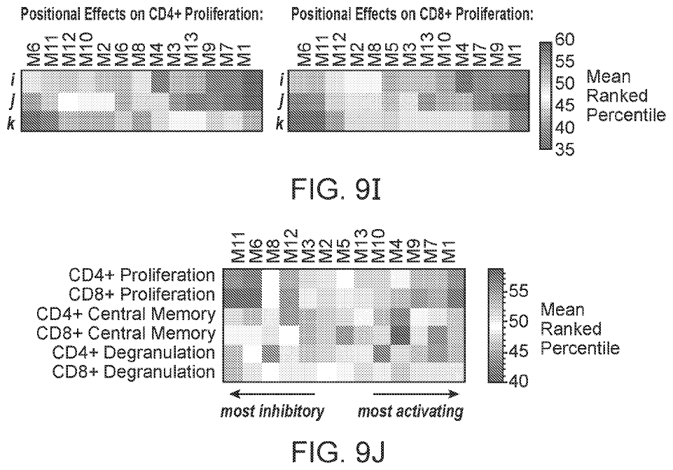

To assess the overall contribution of individual motifs, all the CARs in the library were ranked by neural network predicted cytotoxicity and stemness and then assessed whether motifs were enriched in the strong or weak ends of the phenotypic distribution (FIG. 3A, 8). If a motif is generally activating for a phenotype, then it is expected to be more common in highly ranked CARs; if a motif is inhibitor it is expected to be more common in poorly ranked CARs. Although the effects observed in this distribution analysis depend on other motifs in the CAR and the position of the motif in question, the distributions are informative of the overall effect each motif has in the context of the library. An analogous distribution analysis was also performed on the pooled screening proliferation data (FIG. 9).

This distribution analysis highlighted several potent motifs know to play activating and inhibitory roles. For example motif M9 is the PVQE (SEQ ID NO: 15) motif (from CD40) that binds TRAF2, and is associated with T cell activation and function(22, 23). Accordingly, M9 is enriched in CARs with high cytotoxicity (mean 66 percentile) and high sternness (mean 63 percentile), indicating that overall it promotes both of these phenotypes. In a contrasting example, M6 (from LAIR1) recruits the phosphatase SHP-1, a potent inhibitor of T cell activation. Accordingly. M6 is enriched in CARs with low cytotoxicity (mean 36 percentile) and low stemness (mean 45 percentile), indicative of inhibition of both phenotypes. Interestingly, some motifs can activate one phenotype and inhibit another: M5, which binds Vav1, is unrepresented in CARs with high cytotoxicity (mean 25 percentile), but overrepresented in CARs with high sternness (mean 64 percentile). These results suggest the unexpected finding that Vav1 signaling promotes sternness, while inhibiting killing. The quantified effect of all individual motifs on cytotoxicity and stemness are shown in the heatmap in FIG. 9. The TRAF binding motifs (M9 and M10) are among the best at promoting both cytotoxicity and stemness.

To examine motif pairs that favored particular phenotypes, the occurrence of each possible pair (without regard to order) in the ranked distribution was examined. Several specific motif pairs appear to promote both robust cytotoxicity and sternness when they occur in combination within a costimulatory domain. For example, M1 (PLCγ1) and M10 (TRAF) are each activating with respect to cytotoxicity (means: 58 and 60), but the P1+P10 motif pair is even more strongly activating (mean: 75 percentile). The predicted mean cytotoxicity and stemness percentiles for all 144 pairs of motifs M1-M12 is shown in FIG. 3B. The motif pairs M1+M10, M1+M9, M9+M9, and M9+M10 are best at promoting cytotoxicity and stemness. These pairs all suggest that TRAF-binding motifs (M9 and M10) work well in tandem, as well as in combination with the motif that recruits PLCγ1 (M1) whose signaling is known to activate NFkB. As examined below, it was hypothesized that these pathways are likely to serve complementary roles in these phenotypes. A number of motif pairs strongly inhibit cytotoxicity and stemness. Not surprisingly, all four motif pairs with the lowest cytotoxicity and stemness contain M6 which binds the inhibitory phosphatase SHP-1. Similarly, the tandem occurrence of M5 (Vav1 motif) leads to the strongest combination of stemness with low cytotoxicity.

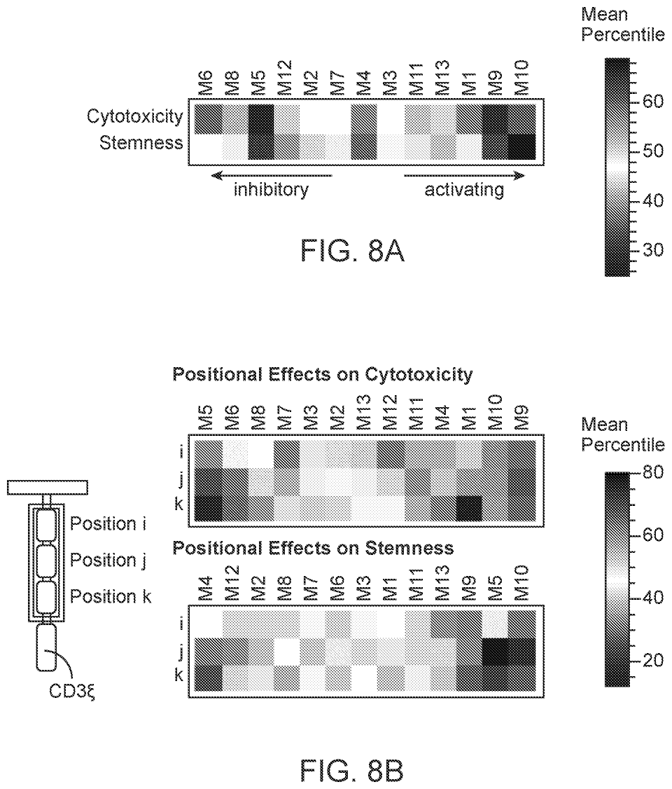

Finally, it was found that the phenotype can vary depending on the position of a motif within the costimulatory domain (FIG. 8. B). For example, M1 (PLCγ1) shows strong cytotoxicity when in positions k or j. and weak cytotoxicity in position i (FIG. 3C). M9 (TRAF) and M10 (TRAF) show optimal cytotoxicity and stemness when in positions i and j. This is consistent with the experimental observation that TRAF-binding parts M9 and M10 followed by M1 (in N- to C-terminal order) promote the most robust cytotoxicity and stemness (M1 followed by M9 or M10 does not (FIG. 6C). These results suggests that shuffling motif position is an approach for calibrating phenotype.

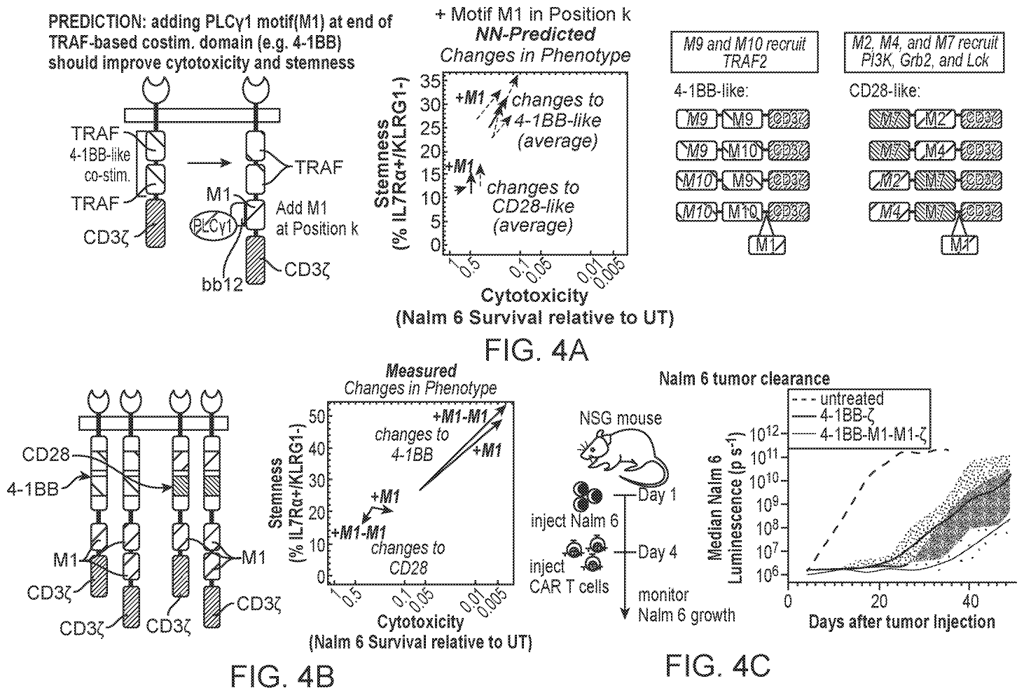

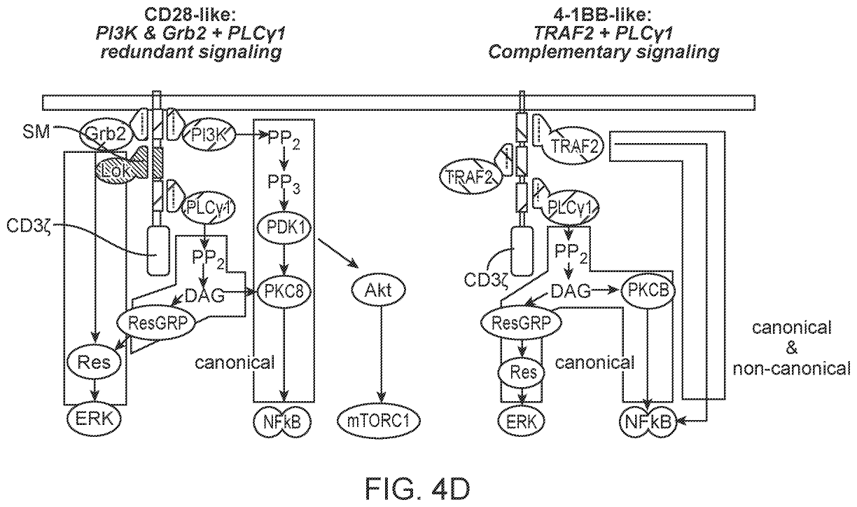

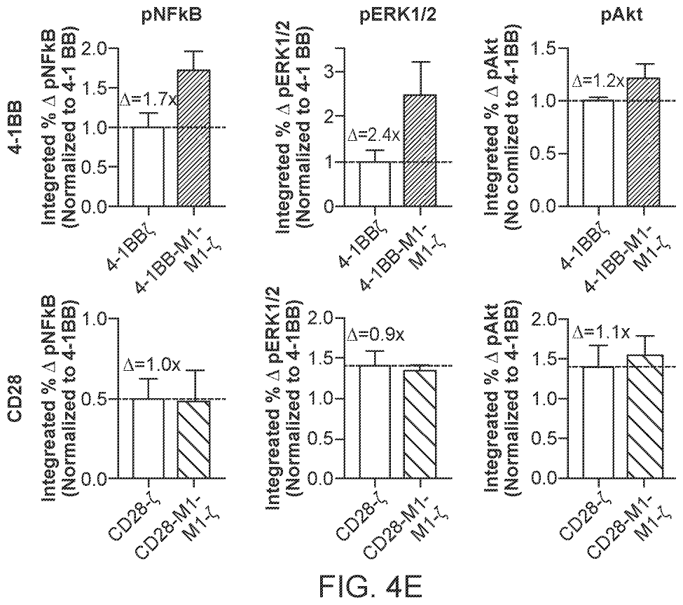

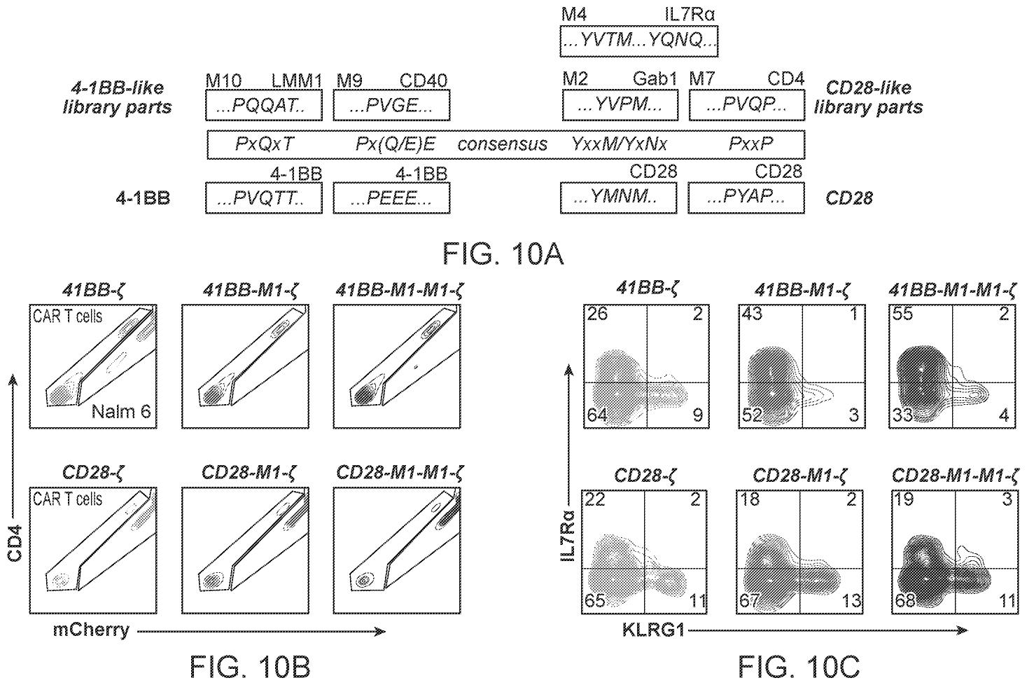

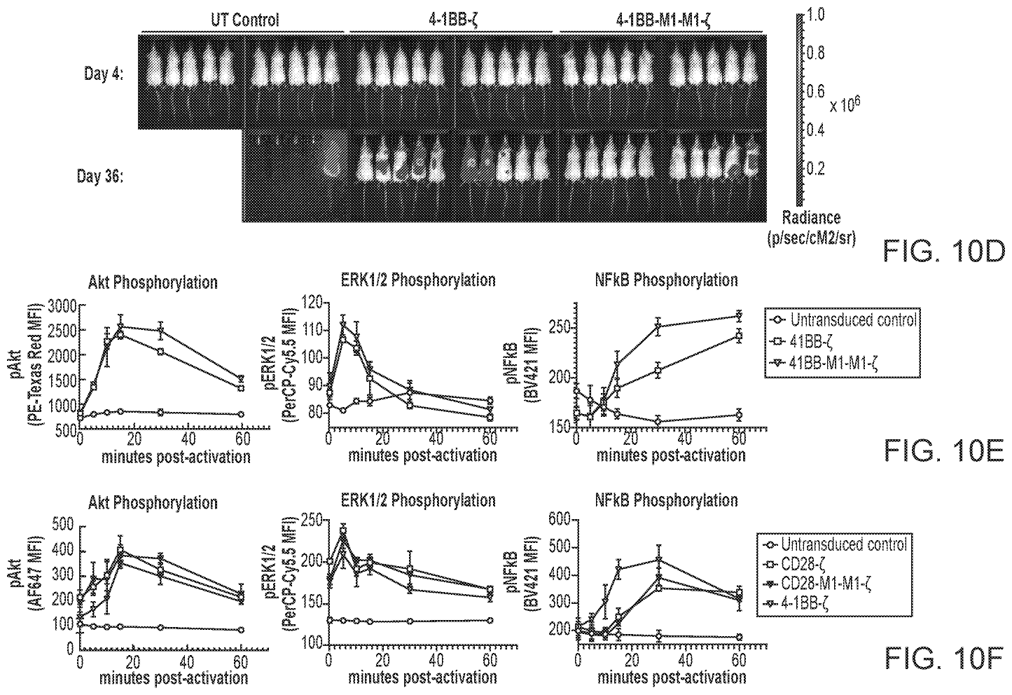

The above distribution analysis quantifies elements of motif language, capturing the effects of motifs (word meaning), motif pairs (word combinations), and motif position (word order) on phenotype. The analysis also yields design rules that can inform combinations and arrangements of motifs capable of producing a desired cell fate. For example, a clearly emergent hypothesis is that a synthetic costimulatory domain which contains one or more TRAF binding motifs (M9 or M10) followed by a PLCγ1 (M1) motif could be highly effective at promoting both cytotoxicity and sternness (FIG. 4A). While tandem TRAF binding motifs occur in the naturally evolved 4-1BB receptor (24) (FIG. 10A), the combination of TRAF and PLCγ1 motifs are not found in natural characterized immune receptors. Thus, adding PLCγ1 (M1) motifs to 4-1BB-like domains were tested to see if they could improve CAR phenotype. Moreover, one can determine if adding M1 might be a general strategy to improve the efficacy of other costimulatory domains, such as CD28.

To explore this hypotheses, the neural network-predicted library was examined to predict the effects of adding the M1 motif to CD28-like and 4-1BB-like synthetic costimulatory domains (library members whose signaling motifs shared the overall configuration of natural signaling motifs in CD28 and 4-1BB) (FIG. 4A). The 4-1BB-like costimulatory domains are predicted by the neural network model to show increased cytotoxicity and stemness, consistent with experimental observations. In contrast, addition of M1 motifs to CD28-like costimulatory domains are not predicted to enhance cytotoxicity or stemness.

Next, to experimentally test the hypothesis, derivatives of the 4-1BB and CD28 costimulatory domains with 1 or 2 copies of the M1 motif added to the C-terminus were synthesized. These costimulatory domains were tested for their ability to kill Nalm 6 and maintain stemness (FIG. 4B). Consistent with predictions, 4-1BB showed significantly enhanced cytotoxicity and stemness upon addition of M1, while CD28 showed almost no change. Significantly, in addition to predicted in vitro changes, the 4-1BB-M1-M1-z CAR construct showed improved efficacy against a Nalm 6 tumor NSG mouse model (FIG. 4C. FIG. 10D). Relative to standard 4-1BB CAR T cells, the 4-1BB-P1-P1-z CAR T cells were able to delay the growth of Nalm 6 for an additional two weeks, validating the predictions from the library/neural network model.

PLCγ1 catalyzes the production of DAG from PIP₂, which activates RasGRP and PKCq. subsequently activating ERK1/2 and NFkB. This signaling is similar and possibly redundant to that of PI3K and Grb2, which also activate RasGRP and PKCq. TRAF signaling, however, does not activate RasGRP or PKCq. such that PLCγ1 and TRAF signaling are likely to be more complementary (FIG. 4D). The 4-1BB-M1-M1-z CAR construct (compared to standard 4-1BB1-ζCAR) was experimentally characterized by measuring the kinetics of Akt, ERK1/2, and NFkB phosphorylation upon Nalm 6 stimulation (FIG. 4E, FIG. 10E). The addition of the M1 motifs increase phosphorylation of ERK1/2 (1.7-fold) and NFkB (2.4-fold), both of which are downstream of PLCγ1 activity. Phosphorylation of Akt, which is not downstream of PLCγ1 activity, shows only a 1.2-fold increase. The observed increase in NFkB and ERK1/2 activation support the hypothesis that PLCγ1 signaling is complementary to TRAF signaling and is consistent with previous reports that NFkB activation is important for the maintenance of CD8+ T cell memory(25). In contrast, no significant increase in NFkB and ERK1/2 activation is observed for a CAR in which the PLCγ1 motif is appended to the CD28 costimulatory domain.

Addition of PLCγ1-binding motifs (P1) to the 4-1BB costimulatory domain increases in vitro killing and stemness of CAR T cells.

Materials and Methods. Primary human T cells transduced with CAR library constructs were mixed with Nalm 6 to reach 1×10⁶ T cells per mL and 2×10⁶ per mL Nalm6 and centrifuged at 300 g for 2 min. For day 3, 5, and 7 challenges with Nalm 6, 80 μL of co-cultured T cells and Nalm6 were added to 120 μL of Nalm 6 at 2×10⁶ per mL and centrifuged at 300 g for 2 min.

For analysis of cell surface receptor expression, samples were centrifuged at 500×g for 5 min and resuspended in a 50 μL volume with the appropriate antibodies diluted 1:50 in calcium-free magnesium-free PBS with 5% FBS and 5 mM EDTA. After a 30-min incubation at room temperature, samples were washed twice calcium-free magnesium-free PBS with 5% FBS and 5 mM EDTA. (FBS; UCSF Cell Culture Facility). Samples were analyzed for protein expression on a BD LSRII. Antibodies are as follows: APC Mouse anti-human KLRG1 clone SA231A2 (BioLegend #367716), BV421 Mouse anti-human IL7Ra clone HIL-7R-M21 (BD Biosciences #562436), BV786 Mouse anti-human CD62L clone SK11 (BD Biosciences #565311), AF700 Mouse anti-human CD45RA clone HI100 (BD Biosciences #560673), PE Mouse anti-human CD4 clone RPA-T4 (BD Biosciences #555347).

Primary human T cells transduced with CAR library constructs were mixed with Nalm 6 to reach 1×10⁶ T cells per mL and 2×10⁶ per mL Nalm6 and centrifuged at 300 g for 2 min. For day 3, 5, and 7 challenges with Nalm 6, 80 μL of co-cultured T cells and Nalm6 were added to 120 μL of Nalm 6 at 2×10⁶ per mL and centrifuged at 300 g for 2 min.

For analysis of cell surface receptor expression, samples were centrifuged at 500×g for 5 min and resuspended in a 50 μL volume with the appropriate antibodies diluted 1:50 in calcium-free magnesium-free PBS with 5% FBS and 5 mM EDTA. After a 30-min incubation at room temperature, samples were washed twice calcium-free magnesium-free PBS with 5% FBS and 5 mM EDTA. (FBS; UCSF Cell Culture Facility). Samples were analyzed for protein expression on a BD LSRII. Antibodies are as follows: APC Mouse anti-human KLRG1 clone SA231A2 (BioLegend #367716), BV421 Mouse anti-human IL7Ra clone HIL-7R-M21 (BD Biosciences #562436).

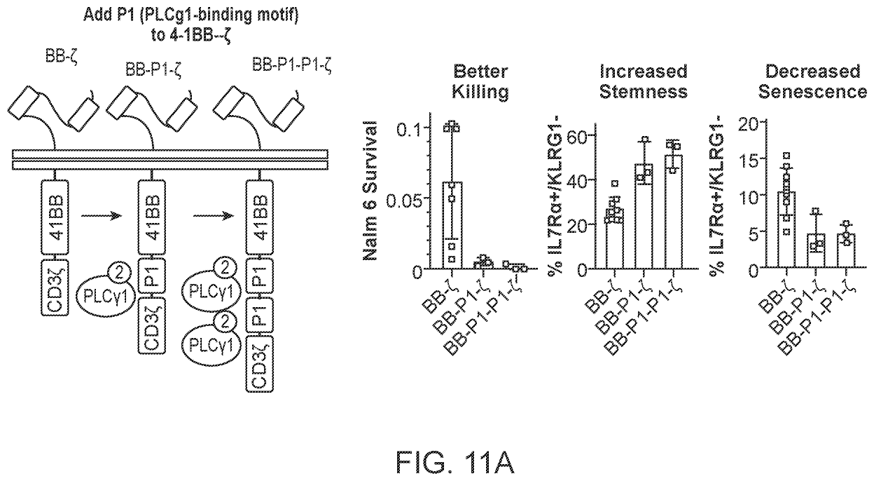

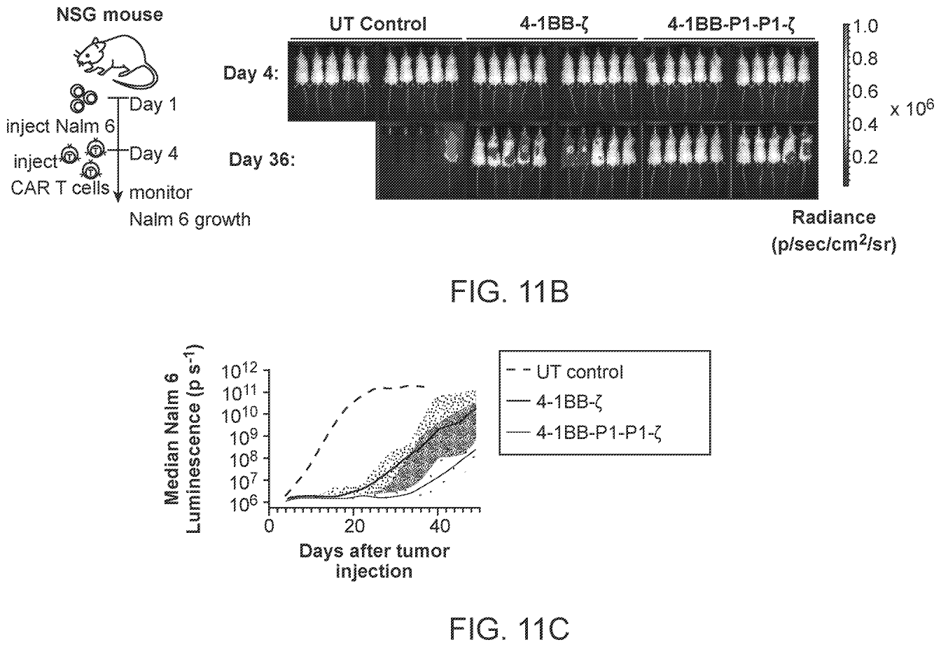

Results. Addition of P1 increased the killing and stemness of CARs with 4-1BB signaling domains. A second copy of P1 slightly increased the stemness relative to a single copy. Addition of P1 also reduced the population of IL7Rα−/KLRG1+ cells, which have the opposite expression of the stem-like population and may be losing beneficial stem-like properties. These results are shown in FIG. 11A.

These results show that addition of PLCγ1-binding motifs to 4-1BB signaling domains enhances in vitro anti-tumor efficacy of CAR T cells by increasing killing and stemness.

CAR T cells with 4-1BB-P1-P1-ζ signaling domains showed improved tumor control relative to 4-1BB-ζ.

Materials and Methods. All mouse experimental procedures were conducted according to Institutional Animal Care and Use Committee (IACUC)-approved protocols. Female immunocompromised NOD-SCID-II2rg^{-/-} (NSG) mice were obtained from UCSF breeding core. On Day 1, mice were inoculated with 0.5×10⁶ Nalm6 leukemia via tail vein injection. On Day 4, mice were injected with 3×10⁶ T cells via tail vein injection. Leukemia progression was measured by bioluminescent imaging using the IVIS 100 (Xenogen) preclinical imaging system. Images were acquired 15 minutes following intraperitoneal (i.p.) injection with 150 mg/kg of D-luciferin (Gold Technology #LUCK-100). Display and adjustment of bioluminescence intensities was performed using the Living image 4.5.4 software (Perkin Elmer). Mice were humanely euthanized when IACUC-approved endpoint (hunching, neurological impairments such as circling, ataxia, paralysis, limping, head tilt, balance problems, seizures, tumor volume burden) was reached (n=5 to 10 mice per group).

The amino acid sequence of the 4-1BB-P1-P1-ζ intracellular signaling domain is shown below, with relevant parts indicated.

Full Sequence:

(SEQ ID NO: 1)
KRGRKKLLYIFKQPFMRPVQTTQEEDGCSCRFPEEEEGGCELGSG
SGSDYHNPGYLVVLPDSTPGSGSGSDYHNPGYLVVLPDSTPGSGS
GSRVKFSRSADAPAYKQGQNQLYNELNLGRREEYDVLDKRRGRDP
EMGGKPRRKNPQEGLYNELQKDKMAEAYSEIGMKGERRRGKGHDG
LYQGLSTATKDTYDALHMQALPPRS

Subsequences in order (from membrane inward):

4-1BB ICD:
(SEQ ID NO: 34)
KRGRKKLLYIFKQPFMRPVQTTQEEDGCSCRFPEEEEGGCEL
P1:
(SEQ ID NO: 20)
DYHNPGYLVVLPDSTP
P1
(SEQ ID NO: 20)
DYHNPGYLVVLPDSTP
CD3ζ:
(SEQ ID NO: 35)
RVKFSRSADAPAYKQGQNQLYNELNLGRREEYDVLDKRRGRDP
EMGGKPRRKNPQEGLYNELQKDKMAEAYSEIGMKGERRRGKGH
DGLYQGLSTATKDTYDALHMQALPPRS

Sequences for Parts of Interest from Library:

P1-
(SEQ ID NO: 20)
DYHNPGYLVVLPDSTP
(binds to PLCγ1)
P4-
(SEQ ID NO: 28)
LGSNQEEAYVTMSSFYQNQ
(binds to PI3K/Grb2)
P9-
(SEQ ID NO: 22)
GSNTAAPVQETLHGCQ
(binds to TRAF2)
P10-
(SEQ ID NO: 23)
DDSLPHPQQATDDSGHES
(binds to TRAF2)
P11-
(SEQ ID NO: 24)
KAPHAKQEPQEINFPDDLP
(binds to TRAF6)

All of the subsequences in the intracellular signaling domains are separated by GSGSGS (SEQ ID NO: 36) linkers. These linkers are not believed to be essential for these molecules to work, since they are simply left-over sequences from construct assembly.

Results. 4-1BB-P1-P1-ζ reduced overall tumor burden compared to 4-1BB-ζ. Initial tumor control was prolonged, and median tumor burden was approximately 50-100 fold lower for the 4-1BB-P1-P1-ζ construct. These results are shown in FIGS. 11B and C.

These results show that addition of PLCγ1-binding motifs to 4-1BB signaling domains enhances anti-tumor efficacy of CAR T cells.

The synthetic sequences should work in other combinations, with other co-stimulatory domains. For example, several co-stimulatory domains should be improved by the addition of PLCγ1-binding motif such as P1, especially co-stimulatory domains that recruit TRAFs. These include natural co-stimulatory domains from 4-1BB, CD40, BAFF-R and other TNFR family receptors, as well as synthetic constructs such as the P11-P10-P10, which is essentially a synthetic derivative of CD40. The P11-P10-P10 contains three TRAF-binding motifs.

### REFERENCES

1. D. M. Barrett, N. Singh, D. L. Porter, S. A. Grupp, C. H. June, Chimeric antigen receptor therapy for cancer. Annu Rev Med 65, 333-347 (2014).
2. B. A. Irving, A. Weiss, The cytoplasmic domain of the T cell receptor zeta chain is sufficient to couple to receptor-associated signal transduction pathways. Cell 64, 891-901 (1991).
3. C. Romeo, B. Seed, Cellular immunity to HIV activated by CD4 fused to T cell or Fc receptor polypeptides. Cell 64, 1037-1046 (1991).
4. F. Letourneur, R. D. Klausner, T-cell and basophil activation through the cytoplasmic tail of T-cell-receptor zeta family proteins. Proc Natl Acad Sci USA 88, 8905-8909 (1991).
5. A. Krause et al., Antigen-dependent CD28 Signaling Selectively Enhances Survival and Proliferation in Genetically Modified Activated Human Primary T Lymphocytes. Journal of Experimental Medicine 188, 619-626 (1998).
6. H. M. Finney, A. D. G. Lawson, C. R. Bebbington, A. N. C. Weir, Chimeric Receptors Providing Both Primary and Costimulatory Signaling in T Cells from a Single Gene Product. The Journal of Immunology 161, 2791-2797 (1998).
7. C. Imai et al., Chimeric receptors with 4-1BB signaling capacity provoke potent cytotoxicity against acute lymphoblastic leukemia. Leukemia 18, 676-684 (2004).
8. C. A. Ramos et al., In Vivo Fate and Activity of Second- versus Third-Generation CD19-Specific CAR-T Cells in B Cell Non-Hodgkin's Lymphomas. Mol Ther 26, 2727-2737 (2018).
9. G. Enblad et al., A Phase I/IIa Trial Using CD19-Targeted Third-Generation CAR T Cells for Lymphoma and Leukemia. Clin Cancer Res 24, 6185-6194 (2018).
10. Y. Kagoya et al., A novel chimeric antigen receptor containing a JAK-STAT signaling domain mediates superior antitumor effects. Nat Med 24, 352-359 (2018).
11. C. A. Koch, D. Anderson, M. F. Moran, C. Ellis, T. Pawson, SH2 and SH3 Domains: Elements that Control Interactions of Cytoplasmic Signaling Proteins. Science 252, 668-674 (1991).
12. M. Sudol, From Src Homology domains to other signaling modules: proposal of the ‘protein recognition code’. Oncogene 17, 1469-1474 (1998).
13. O. U. Kawalekar et al., Distinct Signaling of Coreceptors Regulates Specific Metabolism Pathways and Impacts Memory Development in CAR T Cells. Immunity 44, 380-390 (2016).
14. T. Kyung et al., bioRxiv 2021.07.09.450900.
15. D. B. Goodman et al., bioRxiv 2021.071451980
16. R. B. Di Roberto et al., bioRxiv 2021.08.23.457342
17. H. Dinkel et al., ELM—the database of eukaryotic linear motifs. Nucleic Acids Res 40, D242-251 (2012).
18. J. C. D. Houtman et al., Binding Specificity of Multiprotein Signaling Complexes Is Determined by Both Cooperative Interactions and Affinity Preferences. Biochemistry 43, 4170-4178 (2004).
19. M. Xu, R. Zhao, Z. J. Zhao, Identification and characterization of leukocyte-associated Ig-like receptor-1 as a major anchor protein of tyrosine phosphatase SHP-1 in hematopoietic cells. J Biol Chem 275, 17440-17446 (2000).
20. M. Borsa et al., Modulation of asymmetric cell division as a mechanism to boost CD8+ T cell memory. Science Immunology 4, eaav1730 (2019).
21. D. Herndler-Brandstetter et al., KLRG1(+) Effector CD8(+) T Cells Lose KLRG1, Differentiate into All Memory T Cell Lineages, and Convey Enhanced Protective Immunity. Immunity 48, 716-729 e718 (2018).
22. S. P. Schoenberger, R. E. M. Toes, E. I. H. van der Voort, R. Offringa, C. J. M. Melief, T-cell help for cytotoxic T lymphocytes is mediated by CD40-CD40L interactions. Nature 393, 480-483 (1998).
23. S. M. McWhirter et al., Crystallographic analysis of CD40 recognition and signaling by human TRAF2. Proceedings of the National Academy of Sciences of the United States of America 96, 8408-8413 (1999).
24. H. H. Park, Structure of TRAF Family: Current Understanding of Receptor Recognition.

Front Immunol 9, 1999 (2018).
25. K. M. Knudson et al., NFkappaB-Pim-1-Eomesodermin axis is critical for maintaining CD8 T-cell memory quality. Proc Natl Acad Sci USA 114, E1659-E1667 (2017).
26. Y. Jung, J. Hu, A K-fold averaging cross-validation procedure. Journal of Nonparametric Statistics 27, 167-179 (2015).
27. A. Géron, Hands-on machine learning with Scikit-Learn, Keras, and TensorFlow: Concepts, tools, and techniques to build intelligent systems. (O'Reilly Media, 2019).
28. S. Wang et al., Massive computational acceleration by using neural networks to emulate mechanism-based biological models. Nat Commun 10, 4354 (2019).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An engineered immune receptor, comprising:
(a) an extracellular binding domain that binds to a protein on the surface of another cell;
(b) a transmembrane domain; and
(c) a co-stimulatory domain that comprises one or more TRAF protein binding motifs and one or more PLCγ1-binding motifs.

2. The receptor of claim 1, wherein one or more PLCγ1 (phospholipase C gamma)-binding motifs comprise one or more PLCγ1-binding motifs of the consensus sequence (Y)[AFILVWY]x[AFILVWY].

3. The receptor of claim 2, wherein the PLCγ1-binding motif is YLVV (SEQ ID NO: 6), YIIP (SEQ ID NO: 2), YLIP (SEQ ID NO: 3), YLRV (SEQ ID NO: 4) or YLVP (SEQ ID NO: 5).

4. The receptor of any prior claim, wherein the TRAF protein binding motif is PVQE (SEQ ID NO: 15), PQQAT (SEQ ID NO: 16) or PQEINF (SEQ ID NO: 17).

5. The receptor of claim 1, wherein the receptor further comprises:
(d) a T cell activation domain and
wherein the receptor is a chimeric antigen receptor (CAR).

6. The receptor of claim 5, wherein T cell activation domain comprises at least one immunoreceptor tyrosine-based activation motif (ITAM).

7. The receptor of claim 1, wherein the receptor further comprises a co-stimulatory domain from 4-1BB (CD137), CD28, ICOS, OX-40, BTLA, CD27, CD30, GITR, CD40, CD40L, HVEM, or a TLR, or a variant thereof.

8. The receptor of claim 1, wherein the extracellular binding domain comprises the antigen binding domain of a nanobody or a scFv.

9. An engineered immune receptor, comprising:
(a) an extracellular binding domain that binds to a protein on the surface of another cell;
(b) a transmembrane domain; and
(c) one or more synthetic co-stimulatory motifs that bind to a protein listed in FIG. 1, or any combination thereof.

10. The receptor of claim 9, wherein the receptor further comprises:
(d) a T cell activation domain and
wherein the receptor is a chimeric antigen receptor (CAR).

11. The receptor of claim 10, wherein T cell activation domain comprises at least one immunoreceptor tyrosine-based activation motif (ITAM).

12. The receptor of claim 11, wherein the ITAM is from DAP12; FCER1G (Fc epsilon receptor I gamma chain); CD3D (CD3 delta); CD3E (CD3 epsilon); CD3G (CD3 gamma); CD3Z (CD3 zeta); or CD79A (antigen receptor complex-associated protein alpha chain), or a variant thereof.

13. The receptor of claim 9, wherein the engineered immune receptor:
(d) does not comprise a T cell activation domain and
wherein the receptor is a chimeric costimulatory receptor (CCR).

14. The receptor of any of claims 9-13, wherein the one or more synthetic co-stimulatory motifs include one or more PLCγ1-binding motifs.

15. The receptor of claim 14, wherein one or more PLCγ1 (phospholipase C gamma)-binding motifs comprise one or more PLCγ1-binding motifs of the consensus sequence (Y)[AFILVWY]x[AFILVWY].

16. The receptor of any of any of claims 14-15, wherein PLCγ1-binding motifs is YLVV (SEQ ID NO: 6), YIIP (SEQ ID NO: 2), YLIP (SEQ ID NO: 3), YLRV (SEQ ID NO: 4) or YLVP (SEQ ID NO: 5).

17. The receptor of any of claims 9-16, wherein the one or more synthetic co-stimulatory motifs include one or more TRAF protein binding motifs.

18. The receptor of any of claims 9-17, wherein the one or more synthetic co-stimulatory motifs include one or more motifs of the consensus sequence;
(i) Px(Q/E)E, Px(Q/E)xxD or Px(Q/E)xT, where x is any amino acid;
(ii) Arg-Leu-X-Ala, where X is be any amino acid and Ala can be replaced by a small uncharged residue; or
(iii) PxExxZ, where x is any amino acid and Z is acidic or aromatic amino acid.

19. The receptor of any of claims 9-18, wherein one or more synthetic co-stimulatory motifs include one or more of PVQE (SEQ ID NO: 15), PQQAT (SEQ ID NO: 16) and PQEINF(SEQ ID NO: 17).

20. The receptor of any of claims 9-19, wherein the one or more synthetic co-stimulatory motifs include one or more TRAF protein binding motifs and one or more PLCγ1-binding motifs.

21. The receptor of any of claims 9-20, wherein the receptor further comprises a co-stimulatory domain from 4-1BB (CD137), CD28, ICOS, OX-40, BTLA, CD27, CD30, GITR, CD40, CD40L, HVEM, or a TLR, or a variant thereof.

22. The receptor of any of claims 9-21, wherein the extracellular binding domain comprises the antigen binding domain of a nanobody or scFv, a ligand for a receptor, or a receptor for a ligand.

23. A nucleic acid encoding the engineered immune receptor of any of claims 1-22.

24. An immune cell expressing the engineered immune receptor of any of claims 1-22, wherein binding of the engineered immune receptor to the protein on the surface of the other cell activates the immune cell.

25. The immune cell of claim 24, wherein the cell is a myeloid or lymphoid cell.

26. The immune cell of claim 25, wherein the lymphoid cell a T lymphocyte, a B lymphocyte or a Natural Killer cell.

27. A method of treating a subject for a disease, the method comprising:
administering to the subject a cell of any of claims 24-26.

28. The method of claim 27, wherein the disease is cancer.


SEQUENCE LISTING (36 sequences)

SEQ ID NO: 1 - protein, 205 aa, Homo sapiens
KRGRKKLLYIFKQPFMRPVQTTQEEDGCSCRFPEEEEGGCELGSGSGSDYHNPGYLVVLPDSTPGSGSGSDYHNPGYLVVLPDSTPGSGSGSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRS

SEQ ID NO: 2 - protein, 4 aa, Homo sapiens
YIIP

SEQ ID NO: 3 - protein, 4 aa, Homo sapiens
YLIP

SEQ ID NO: 4 - protein, 4 aa, Homo sapiens
YLRV

SEQ ID NO: 5 - protein, 4 aa, Homo sapiens
YLVP

SEQ ID NO: 6 - protein, 4 aa, Homo sapiens
YLVV

SEQ ID NO: 7 - protein, 4 aa, Homo sapiens
YVPM

SEQ ID NO: 8 - protein, 4 aa, Homo sapiens
YENL

SEQ ID NO: 9 - protein, 4 aa, Homo sapiens
YVTM

SEQ ID NO: 10 - protein, 4 aa, Homo sapiens
YQNQ

SEQ ID NO: 11 - protein, 4 aa, Homo sapiens
YESP

SEQ ID NO: 12 - protein, 6 aa, Homo sapiens
ITYAAV

SEQ ID NO: 13 - protein, 4 aa, Homo sapiens
PVQP

SEQ ID NO: 14 - protein, 5 aa, Homo sapiens
RSTKP

SEQ ID NO: 15 - protein, 4 aa, Homo sapiens
PVQE

SEQ ID NO: 16 - protein, 5 aa, Homo sapiens
PQQAT

SEQ ID NO: 17 - protein, 6 aa, Homo sapiens
PQEINF

SEQ ID NO: 18 - protein, 7 aa, Homo sapiens
RPTAVEG

SEQ ID NO: 19 - protein, 18 aa, Artificial Sequence
  Synthetic sequence
SAGSAGSAGSAGSAGSAG

SEQ ID NO: 20 - protein, 16 aa, Homo sapiens
DYHNPGYLVVLPDSTP

SEQ ID NO: 21 - protein, 17 aa, Homo sapiens
LPTWSTPVQPMALIVLG

SEQ ID NO: 22 - protein, 16 aa, Homo sapiens
GSNTAAPVQETLHGCQ

SEQ ID NO: 23 - protein, 18 aa, Homo sapiens
DDSLPHPQQATDDSGHES

SEQ ID NO: 24 - protein, 19 aa, Homo sapiens
KAPHAKQEPQEINFPDDLP

SEQ ID NO: 25 - protein, 19 aa, Homo sapiens
GSGPGSRPTAVEGLALGSS

SEQ ID NO: 26 - protein, 16 aa, Homo sapiens
EELDENYVPMNPNSPP

SEQ ID NO: 27 - protein, 16 aa, Homo sapiens
EEGAPDYENLQELNHP

SEQ ID NO: 28 - protein, 19 aa, Homo sapiens
LGSNQEEAYVTMSSFYQNQ

SEQ ID NO: 29 - protein, 17 aa, Homo sapiens
PAPSIDRSTKPPLDRSL

SEQ ID NO: 30 - protein, 19 aa, Homo sapiens
LPMDTEVYESPFADPEEIR

SEQ ID NO: 31 - protein, 18 aa, Homo sapiens
KPMAESITYAAVARHSAG

SEQ ID NO: 32 - DNA, 60 bp, Artificial Sequence
  Synthetic sequence
  misc_feature (34)..(38): n is a, c, g, or t
tcgtcggcagcgtcagatgtgtataagagacagnnnnnactggttatcaccctttactgc

SEQ ID NO: 33 - DNA, 56 bp, Artificial Sequence
  Synthetic Sequence
  misc_feature (35)..(39): n is a, c, g, or t
gtctcgtgggctcggagatgtgtataagagacagnnnnncttgtaggcgggagcat

SEQ ID NO: 34 - protein, 42 aa, Homo sapiens
KRGRKKLLYIFKQPFMRPVQTTQEEDGCSCRFPEEEEGGCEL

SEQ ID NO: 35 - protein, 113 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRS

SEQ ID NO: 36 - protein, 6 aa, Artificial Sequence
  Synthetic sequence
GSGSGS